(19) United States
(12) Patent Application Publication
(10) Pub. No.: US 2005/0177224 A1
(43) Pub. Date: Aug. 11, 2005

(54) Vascular fixation device and method

(76) Inventors: Thomas J. Fogarty, Portola Valley, CA (US); D. Bruce Modesitt, San Carlos, CA (US); Neil Holmgren, Chicago, IL (US); Jamie Juliana van Hoften, Lafayette, CA (US); Michael J. Drews, Sacramento, CA (US)

Correspondence Address: DAVID A. LEVINE, P. O. BOX 61180, PALO ALTO, CA, 94306-1180, US

(21) Appl. No.: 10/776,570

(22) Filed: Feb. 11, 2004

(51) Int. Cl. A61F 2/06

(52) U.S. Cl. 623/1.35; 623/1.36; 606/151

(57) ABSTRACT

An intravascular anchoring implant is disclosed. The present invention also relates to the attachment to the intravascular implant of second and possibly third implants, such as a graft attachment device and a vascular graft. Methods of using the implant within the vasculature of the body, particularly adjacent to vascular aneurysms, are also disclosed

### BACKGROUND OF THE INVENTION

1. Field of the Invention

The present invention relates generally to an intravascular fixation implant and methods of using the implant within the vasculature of the body, particularly adjacent to vascular aneurysms. The present invention also relates to the attachment to the intravascular implant of second and possibly third implants, such as a graft attachment device and a vascular graft.

2. Description of the Related Art

An aneurysm is an abnormal dilatation of a biological vessel. Aneurysms can alter flow through the affected vessel and often decrease the strength of the vessel wall, thereby increasing the vessel's risk of rupturing at the point of dilation or weakening. Implanting a vascular prosthesis through the vessel with the aneurysm is a common aneurysm therapy. Vascular grafts and stent grafts (e.g., ANEURX® Stent Graft System from Medtronic AVE, Inc., Santa Rosa, Calif.) are examples of vascular prostheses used to treat aneurysms by reconstructing the damaged vessel.

Stent grafts rely on a secure attachment to the proximal, or upstream, neck of an aneurysm, particularly for aortic abdominal aneurysms (AAA), but several factors can interfere with this attachment. The neck does not contract and expand evenly as blood flows through the vessel. The portion of the neck closest to the spine remains relatively fixed while the remainder of the vessel expands and contracts in response to the changing blood pressure during normal pulsatile flow. This circumferentially dynamic expansion and contraction of the neck presents problems for attachment systems that expand and contract evenly around the entire circumference.

Devices have been developed that attempt to solve the issue of vascular graft attachment, but those that permit for substantial radial expansion and contraction fail to have expansion and contraction rates that vary with respect to the angle around the vessel. U.S. Pat. No. 6,152,956 to Pierce discloses a radially expandable collar connected by wires to an expandable stent. The stent is used to anchor the collar to the aneurysm neck and has barbs with sharp ends that spring radially outward to embed into the walls of the vascular tissue. The stent is expandable, but is equally resilient at all angles around the entire circumference of the stent. Therefore, the stent is not designed to contract and expand dynamically with respect to the angle around the vessel. Further, the barbs are equidistantly located around the circumference of the vessel, further impairing circumferentially dynamic expansion and contraction.

U.S. Pat. No. 6,361,556 by Chuter discloses a stent for attaching to grafts, where the stent is connected to an attachment system for anchoring to the vessel. The attaching system has hooks angled toward the graft. The stent is substantially rigid and balloon expandable and therefore maintains a fixed diameter and resists deformation from forces imposed by the vascular environment. The stent is therefore unable to substantially accommodate any expansion and contraction, let alone circumferentially dynamic expansion and contraction. The stent may not seal the graft under changing geometric conditions over time. The stent also has hooks equidistantly located around the circumference of the vessel that, like the barbs of Chuter described infra, further impair circumferentially dynamic expansion and contraction.

There is thus a need for a device and method that can securely anchor a vascular graft within a vessel and adjust to the circumferentially varying contraction and expansion of the anchoring vessel during normal pulsatile flow. A need also exists for a device and method that can adjust to tortuous vasculature.

### BRIEF SUMMARY OF THE INVENTION

A fixation device for implantation in a biological vessel is disclosed. The fixation device has a frame having a longitudinal axis. The frame is configured to expand at variable amounts circumferentially with respect to the longitudinal axis. The frame can have a first section and a second section. The first section can remain fixed with respect to the vessel.

Also disclosed is a vascular fixation device having a first fixation section, a first arm and a second fixation section. The first arm has a first end and a second end. The first end is attached to the first fixation section. The second end of the first arm is attached to the second fixation section.

The vascular fixation device can also have a second arm. The second arm can have a first end and a second end. The first end of the second arm can be attached to the first fixation section. The second end of the second arm can be a terminus. The vascular fixation device can also have a third arm extending from the second fixation section.

A vascular fixation device having a first fixation section, a first arm, and a second arm is also disclosed. The first arm extends from the first fixation section. The first arm has a first end. The first end of the first arm has a terminus. A second arm extends from the first fixation section. The second arm has a first end. The first end of the second arm has a terminus.

The first arm can extend from the fixation section in a first direction. The second arm can extend from the fixation section in a second direction. The first direction can be substantially opposite to the second direction. The device can also have a graft attachment device. The graft attachment device can have a first end and a second end. The first end of the graft attachment device can be attached to the fixation section. The second end of the graft attachment device can be attached to a first vascular graft.

Further disclosed is a device for fixing to a vascular wall. The device has a fixation section, a first arm, a second arm, and a graft attachment device. The first arm extends from a first side of the fixation section. The second arm extends from a second side of the fixation section. The graft attachment device has a first end and a second end. The first end of the graft attachment device is attached to the fixation section.

The second end of the graft attachment device can be attached to a first vascular graft. The first vascular graft can have a bifurcated graft. The second end of the graft attachment device can be attached to a second vascular graft. The first end of the graft attachment device can be attached to the fixation section near the vascular wall. The graft attachment device can be configured to radially expand when the graft attachment device is subject to a force in the direction of the graft.

An assembly for fixing to a vascular wall is also disclosed. The assembly has an anchor and a graft. The graft has a first end. The graft is attached to the anchor. The assembly is configured so that when a force is applied pushing the graft away from the anchor then the first end of the graft radially expands.

Additionally disclosed is a method of attaching a vascular prosthesis to a vascular wall. The method includes deploying a fixation device in a vessel and attaching a vascular prosthesis to the fixation device. The fixation device has a fixation section, a first arm extending from the fixation section, and a second arm extending from the fixation section.

### BRIEF DESCRIPTION OF THE DRAWINGS

FIG. 1 illustrates an embodiment of the intravascular graft anchoring assembly and the see-through proximal end of a graft.

FIGS. 2-4 illustrate various embodiments of vascular fixation devices.

FIGS. 5-7 are top views of various embodiments of vascular fixation devices.

FIGS. 8-16 illustrate various embodiments of vascular fixation devices.

FIG. 17 illustrates one embodiment of a leg.

FIG. 18 illustrates an embodiment of a leg attached to another leg.

FIG. 19 illustrates an embodiment of a leg.

FIGS. 20-22 illustrate various embodiments of the intravascular graft anchoring assembly.

FIG. 23 illustrates an embodiment of a graft attachment device.

FIG. 24 is a top perspective view of an embodiment of a graft attachment device.

FIG. 25 is a front view of the graft attachment device of FIG. 24.

FIG. 26 illustrates an embodiment of a first section of the graft attachment device.

FIG. 27 illustrates an embodiment of a second section of the graft attachment device.

FIG. 28 illustrates an embodiment of a first section of the graft attachment device.

FIG. 29 illustrates an embodiment of a second section of the graft attachment device.

FIG. 30 illustrates an embodiment of a graft attachment device.

FIGS. 31 and 32 illustrate various embodiments of the intravascular graft anchoring assembly.

FIG. 33 illustrates an embodiment of the graft.

FIGS. 34-36 illustrate various embodiments of cross-section A-A of FIG. 33.

FIG. 37 illustrates an embodiment of the rim.

FIGS. 38-40 illustrate various embodiments of cross-section B-B of FIG. 37.

FIG. 41 illustrates an embodiment of the rim.

FIGS. 42-44 illustrate various embodiments of cross-section C-C of FIGS. 41 and 46.

FIG. 45 illustrates an embodiment of the interference receptacle.

FIG. 46 illustrates an embodiment of the rim.

FIG. 47 illustrates an embodiment of the intravascular graft anchoring assembly attached to a graft.

FIG. 48 illustrates an embodiment of the intravascular graft anchoring assembly attached to two grafts.

FIGS. 49-51 are sagittal cross-sections of a method of deploying the intravascular graft anchoring assembly in a patient.

FIG. 52 is cross-section D-D of FIG. 51 during diastole.

FIG. 53 is cross-section D-D of FIG. 51 after diastole and before systole.

FIG. 54 is cross-section D-D of FIG. 51 during systole.

FIGS. 55-57 illustrate a method of using the intravascular graft anchoring assembly of FIG. 32.

FIG. 58 illustrates a method of using the intravascular graft anchoring assembly of FIG. 31.

FIG. 59 is an anterior view of a method of using two intravascular graft anchoring assemblies of FIG. 20.

FIG. 60 is an anterior view of a method of using two intravascular graft anchoring assemblies of FIG. 22.

FIG. 61 illustrates a graft.

FIG. 62 illustrates a method of using the graft.

FIG. 63 illustrates cross-section E-E.

FIGS. 64-71 illustrate various methods of preparing the graft for deployment.

FIGS. 72-84 illustrate various methods of deploying the intravascular graft fixation assembly and the graft.

### DETAILED DESCRIPTION

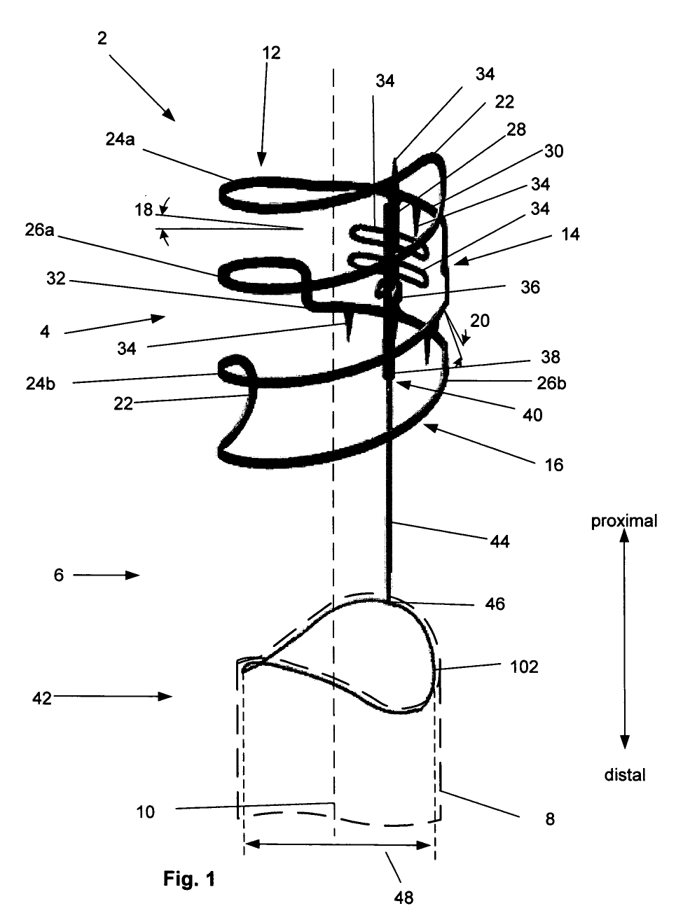

FIG. 1 illustrates an intravascular graft anchoring assembly 2 that can have a vascular fixation device 4 attached to a graft attachment device 6. The graft attachment device 6 can be attached to a graft 8. The intravascular graft anchoring assembly 2 can have a longitudinal axis 10.

The vascular fixation device 4 can be, for example, an AAA anchor, an intravascular stent or a heart valve ring. The vascular fixation device 4 can have a first arm 12 resiliently attached to a fixation section 14 and a second arm 16 resiliently attached to the fixation section 14. The first arm 12 can attach to the opposite side of the fixation section from the second arm 16. The first and second arms 12 and 16 can have a continuously circumferentially expandable spring, for example, a coil spring, angled spring, corrugated sheet, or a combination thereof, or the first arm 12 can be not continuously circumferentially expandable, for example a leaf spring.

The first arm 12 can extend from the fixation section 14 at a first arm angle 18. The first arm angle 18 can be from about −85° to about 85°, more narrowly from about −60° to about 60°, for example about 0°. The second arm 16 can extend from the fixation section 14 at a second arm angle 20. The second arm angle 20 can be from about −85° to about 85°, more narrowly from about −60° to about 60°, for example about 0°.

The first arm 12 can be attached to the fixation section 14. The first arm 12 can have a terminus 22 at the end opposite to the attachment to the fixation section 14. The first arm 12 can have a first member 24a and a second member 26a.

The second arm 16 can be attached to the fixation section 14. The second arm 16 can have a terminus 22 at the end opposite to the attachment to the fixation section 14. The second arm 16 can have a first member 24b and a second member 26b. The first and second members 24b and 26b of the second arm 16 can be integral with or distinct from the first and second members 24a and 26a of the first arm 12. The second arm 16 can be similar to the first arm 12. The first arm 12 can be about parallel with the second arm 16. The first arm 12 can be unparallel with the second arm 16.

The fixation section 14 can have a support structure, for example, a back member 28 attached at one end to a top member 30 and at the opposite end to a bottom member 32. The top member 30 can distinctly or integrally attach to the first members 24 of the first and/or second arms 12 and/or 16. The bottom member 32 can distinctly or integrally attach to the second members 26 of the first and/or second arms 12 and/or 16. The fixation section 14 can have tissue mainstays 34. The tissue mainstays 34 can be, for example, a barb, spike, tab, deflected member, hole in a plate or tab, tissue in-growth matrix, hook, peg, coil, pigtail or leaf spring, or any combination thereof.

The fixation section 14 can have a first and/or second connector 36 and/or 38. The connectors 36 and 38 can be tubes, shafts, weld points, glue, hubs, or any combination thereof. The first and/or second connector 38 can attach directly to the fixation section 14. The second connector 38 can attach to the first connector 36.

The graft attachment device 6 can have a first end 40 that can have one or more legs 44, for example, support wires. The legs 44 can be attached to the first and/or second connectors 36 and/or 38. The legs 44 can extend away from the vascular fixation device 4. The legs 44 can attach to the second end 42 of the graft attachment device 6 at leg attachments 46.

The leg attachments 46 can be integral with, or distinct from, the legs 44. The graft attachment device 6 can have a graft attachment device diameter 48. The graft attachment device diameter 48 can be from about 10 mm (0.39 in.) to about 50 mm (2.0 in.), more narrowly from about 15 mm (0.59 in.) to about 38 mm (1.5 in.). The graft attachment device 6 can be configured so that the graft attachment device diameter 48 can increase, decrease or remain constant when a distally directed force is applied to the graft attachment device 6.

The graft 8 can be fixedly or removably attached to the second end 42 of the graft attachment device 6. The graft 8 can be unitary or bifurcated. The proximal end of the graft 8 can be reinforced to keep open. The graft 8 can be an AV fistula graft, for an abdominal or thoracic aortic aneurysm, for example, TALENT® Stent Graft System and ANEURX® Stent Graft (from Medtronic, Inc., Minneapolis, Minn.), EXCLUDER® (from W.L. Gore & Associates, Inc., Newark, Del.), ANCURE® Endograft System (from Guidant Corp., Indianapolis, Ind.); VANGUARD® stent-graft series and Passager Stent Graft (from Boston Scientific Corp., Natick, Mass.), Lifepath Endovascular Graft (from Edwards Lifescience Corp., Irvine, Calif.), Mialhe/Stentor and Cragg EndoPro System (from MinTec Inc., formerly of France), ZENITH® AAA Endovascular Graft System (from Cook, Inc., Bloomington, Ill.), Quantum (from Johnson & Johnson, New Brunswick, N.J.), POWERLINK® System (from Endologix, Inc., Irvine, Calif.) and C.R. Bard, Inc., Murray Hill, N.J.); Anson (from Anson), ENOVUS (by TriVascular, Inc., Santa Rosa, Calif.), ANACONDA™ Stent-Graft (Sulzer Vascutech, Germany), Corvita Endovascular Graft (from Corvita Inc., Schneider Corp. and Boston Scientific Corp. Natick, Mass.), ELLA Stent-Graft (ELLA-CS, Hradec Králové, Czech Republic) or combinations thereof. The graft 8 can be made from a flexible textile structure, for example, the materials described in the immediately following patents and patent applications, all of which are hereby incorporated by reference in their entirety: U.S. Pat. No. 6,019,786 by Thompson, U.S. Pat. Nos. 6,159,239, 6,164,339, 6,192,994 all by Greenhalgh and U.S. Patent Application Nos. 2002/0083820, 2002/0058992, 2002/0052649, 2002/0052660, 2002/0042644 all by Greenhalgh and 2002/0066360 to Greenhalgh et al.

Any or all elements of the intravascular graft anchoring assembly 2 can be made from, for example, a single or multiple stainless steel alloys, nickel titanium alloys (e.g., Nitinol), cobalt-chrome alloys (e.g., ELGILOY® from Elgin Specialty Metals, Elgin, Ill.; CONICHROME® from Carpenter Metals Corp., Wyomissing, Pa.), molybdenum alloys (e.g., molybdenum TZM alloy, for example as disclosed in International Pub. No. WO 03/082363 A2, published 9 Oct. 2003, which is herein incorporated by reference in its entirety), tungsten-rhenium alloys, for example, as disclosed in International Pub. No. WO 03/082363, polymers such as polyester (e.g., DACRON® from E. I. Du Pont de Nemours and Company, Wilmington, Del.), polypropylene, polytetrafluoroethylene (PTFE), expanded PTFE (ePTFE), polyether ether ketone (PEEK), nylon, polyether-block co-polyamide polymers (e.g., PEBAX® from ATOFINA, Paris, France), aliphatic polyether polyurethanes (e.g., TECOFLEX® from Thermedics Polymer Products, Wilmington, Mass.), polyvinyl chloride (PVC), polyurethane, thermoplastic, fluorinated ethylene propylene (FEP), extruded collagen, silicone, echogenic, radioactive, radiopaque materials or combinations thereof. Examples of radiopaque materials are barium sulfate, titanium, stainless steel, nickel-titanium alloys, tantalum and gold.

Any or all elements of the intravascular graft anchoring assembly 2 can be a matrix for cell ingrowth or used with a fabric, for example a covering (not shown) that acts as a matrix for cell ingrowth. The matrix and/or fabric can be, for example, polyester (e.g., DACRON® from E. I. du Pont de Nemours and Company, Wilmington, Del.), polypropylene, PTFE, ePTFE, nylon, extruded collagen, silicone or combinations thereof.

The elements of the intravascular graft anchoring assembly 2 and/or the fabric can be filled and/or coated with an agent delivery matrix known to one having ordinary skill in the art and/or a therapeutic and/or diagnostic agent. The agents within these matrices can include radioactive materials; radiopaque materials; cytogenic agents; cytotoxic agents; cytostatic agents; thrombogenic agents, for example polyurethane, cellulose acetate polymer mixed with bismuth trioxide, and ethylene vinyl alcohol; lubricious, hydrophilic materials; phosphor cholene; anti-inflammatory agents, for example non-steroidal anti-inflammatories (NSAIDs) such as cyclooxygenase-1 (COX-1) inhibitors (e.g., acetylsalicylic acid, for example ASPIRIN® from Bayer AG, Leverkusen, Germany; ibuprofen, for example ADVIL® from Wyeth, Collegeville, Pa.; indomethacin; mefenamic acid), COX-2 inhibitors (e.g., VIOXX® from Merck & Co., Inc., Whitehouse Station, N.J.; CELEBREX® from Pharmacia Corp., Peapack, N.J.; COX-1 inhibitors); immunosuppressive agents, for example Sirolimus (RAPAMUNE®, from Wyeth, Collegeville, Pa.), or matrix metalloproteinase (MMP) inhibitors (e.g., tetracycline and tetracycline derivatives) that act early within the pathways of an inflammatory response. Examples of other agents are provided in Walton et al, Inhibition of Prostoglandin E₂ Synthesis in Abdominal Aortic Aneurysms, Circulation, Jul. 6, 1999, 48-54; Tambiah et al, Provocation of Experimental Aortic Inflammation Mediators and Chlamydia Pneumoniae, Brit. J. Surgery 88 (7), 935-940; Franklin et al, Uptake of Tetracycline by Aortic Aneurysm Wall and Its Effect on Inflammation and Proteolysis, Brit. J. Surgery 86 (6), 771-775; Xu et al, Sp1 Increases Expression of Cyclooxygenase-2 in Hypoxic Vascular Endothelium, J. Biological Chemistry 275 (32) 24583-24589; and Pyo et al, Targeted Gene Disruption of Matrix Metalloproteinase-9 (Gelatinase B) Suppresses Development of Experimental Abdominal Aortic Aneurysms, J. Clinical Investigation 105 (11), 1641-1649 which are all incorporated by reference in their entireties.

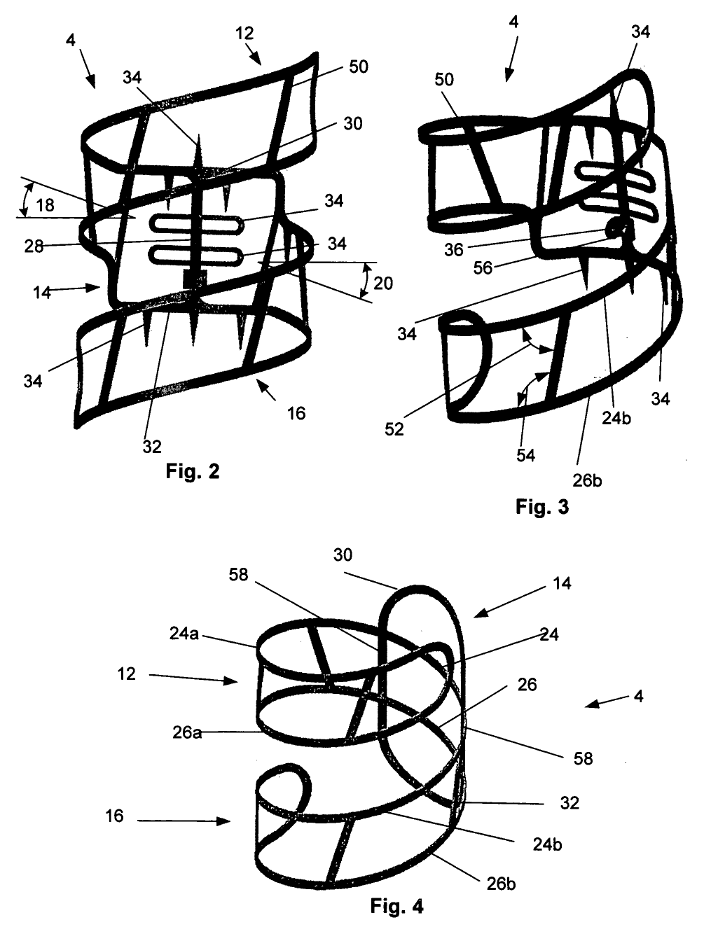

As shown in FIGS. 2 and 3 the first member 24 can be attached to one or more struts 50. One end of the strut 50 can attach to the first member 24 at a first strut angle 52, and the opposite end of the strut 50 can attach to the second member 26 at a second strut angle 54. The first strut angle 52 can be acute, obtuse or right. The second strut angle 54 can be a function of the first strut angle 52, the appropriate arm angle 18 or 20, and the shape of the strut 50. The first member 24 can attach to the second member 26 at the terminus 22 directly or via one or more struts 50. The intravascular graft anchoring assemblies 2 can have no struts 50, as shown in FIG. 1. The first member 24 can be unattached to the second member 26 at the terminus 22 (not shown).

The mainstays 34 can be arranged in various configurations. For example, a single mainstay 34, such as a spike, can extend proximally from the top member 30 and two other mainstays 34, such as spikes, can extend distally from the top member 30. In another example, three mainstays 34 can extend distally from the bottom member 32. In yet another example, two mainstays 34, such as tabs with holes, can extend laterally from the back member 28. In a further example, any combination of the three examples, infra, can be combined. The first and/or second connector 38 can have a pin hole 56 to attach to the legs 44 and/or the second connector 38.

FIG. 4 illustrates the vascular fixation device 4 that can have the fixation section 14 with a rounded or semi-circular shaped top member 30 and/or bottom member 32. Side members 58 can attach the top member 30 and the bottom member 32. The first and second members 24 and 26 of the first and second arms 12 and 16 can be integral. The first and second members 24 and 26 can be distinct from the top member 30 and the bottom member 32.

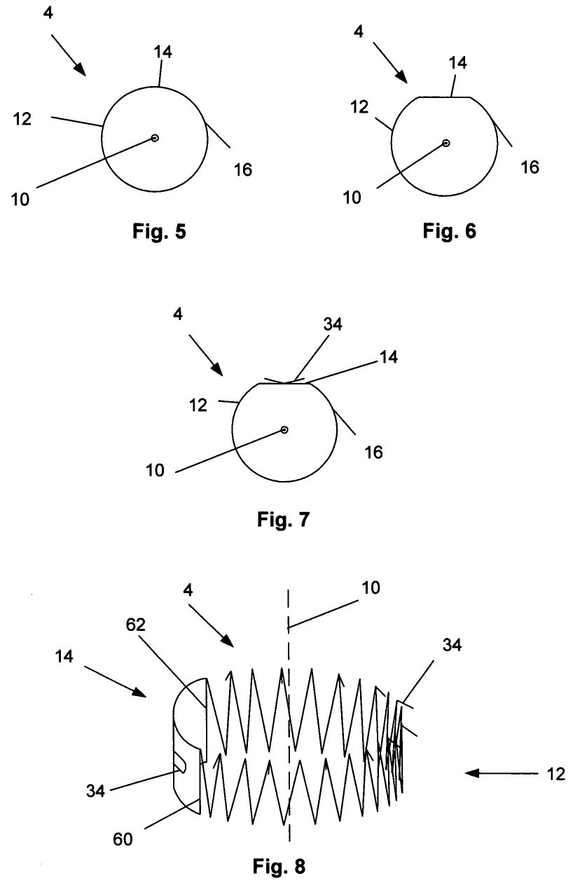

FIGS. 5 through 7 illustrate top views of various vascular fixation devices 4. As shown in FIG. 5, the vascular fixation device 4 can have a round shape, for example a circular or oval shape, with the fixation section 14 similarly curved when viewed from above. As shown in FIG. 6, the fixation section 14 can have an approximately straight shape when viewed from above and the first and second arms 12 and 16 can have a round shape. As shown in FIG. 7, some or all of the mainstays 34 can be directed outward from the fixation section 14 when viewed from above.

FIG. 8 illustrates the vascular fixation device 4 that can have the first arm 12 resiliently attach to the fixation section 14 at a first end 60 of the first arm 12 and a second end 62 of the first arm 12. The first or second end 60 or 62 of the first arm 12 can be unattached to the fixation section 14 and that end 60 or 62 can end in a terminus 22 (not shown). One or more mainstays 34 can extend from the first and/or second arms 12 and/or 16.

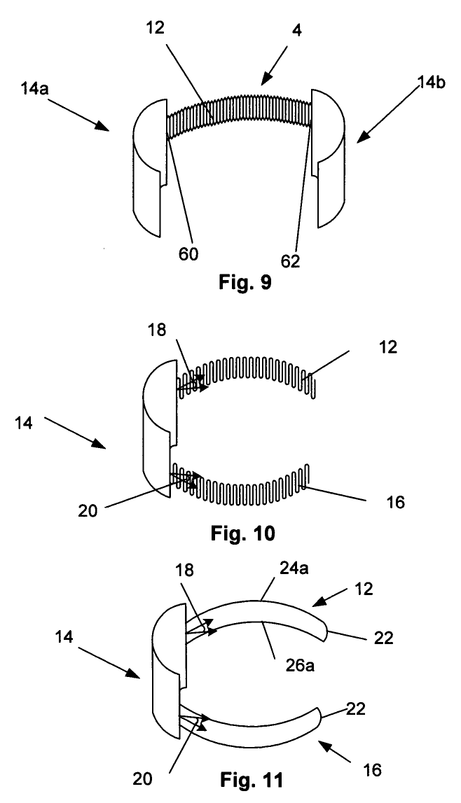

FIG. 9 illustrates the vascular fixation device 4 that can have the first fixation section 14a that can be resiliently attached to the second fixation section 14b. The first end 60 of the first arm 12 can attach to the first fixation section 14a. The second end 62 of the first arm 12 can attach to the second fixation device 14b.

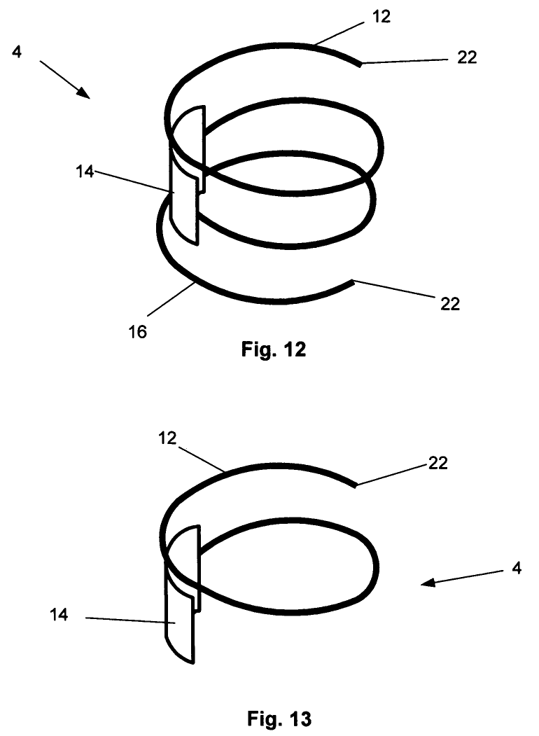

FIGS. 10 through 12 illustrate the vascular fixation device 4 that can have the fixation section 14, the first arm 12 extending from the fixation section 14 and the second arm 16 extending from the fixation section 14. The first arm angle 18 can be equal to the second arm angle 20. The first arm 12 can lie in a plane with the second arm 16, as shown in FIGS. 10 and 11. The arms 12 and 16 can have a sinusoidal configuration, as shown in FIG. 10. The arms 12 and 16 can have first members 24 attached via termini 22 to second members 26, as shown in FIG. 11. The arms 12 and 16 can be individual leaders concluding in their respective termini 22, as shown in FIG. 12. FIG. 13 illustrates the vascular fixation device 4 that can have the first arm 12 extending from the fixation section 14 and concluding in the terminus 22.

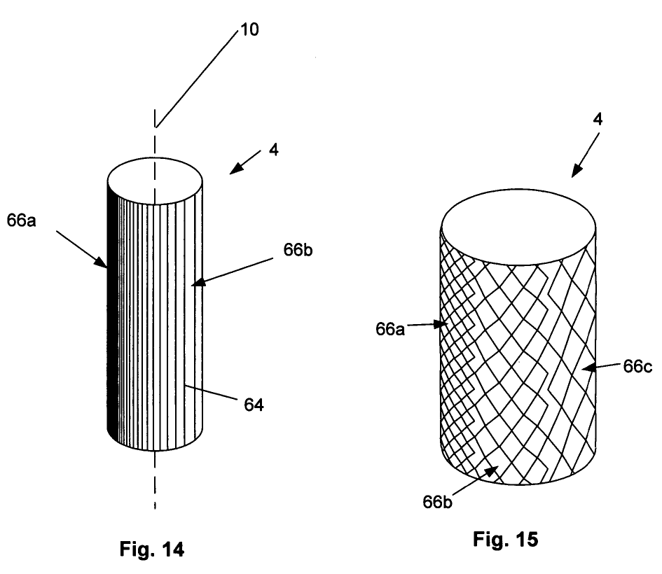

FIG. 14 illustrates the vascular fixation device 4 that can have circumferentially variable amounts of angular expansion when exposed to, or withdrawn from, a radial force with respect to the longitudinal axis 10. Wires or zones 64 can have a resistance to angular expansion. More densely arranged zones 64, for example at a first area 66a, can cause higher resistance to angular expansion. Less densely arranged zones 64, for example at a second area 66b, can cause higher resistance to angular expansion. The zones 64 can be representative of material density, material strength, material type including composite materials, geometric configuration, or combinations thereof. The area with the highest resistance to angular expansion, for example first area 66a, can be the fixation section 14. The vascular fixation device 4 can have one zone 64, two zones 64 or more. The transition between the zones 64 can be gradual or immediate.

FIG. 15 illustrates a wireform or cellular vascular fixation device 4 that can have, for example, three areas 66a, 66b, and 66c. The first area 66a can be the fixation section 14. In the first area 66a, the cells or wireform can be the most densely configured of the three areas 66a, 66b and 66c. The second area 66b can have cells or the wireform of an intermediate density configuration. In the third area 66c, the cells or the wireform can be the least densely configured of the three areas 66a, 66b and 66c. (The top and bottom borders of the vascular fixation device are shown for illustrative purposes.)

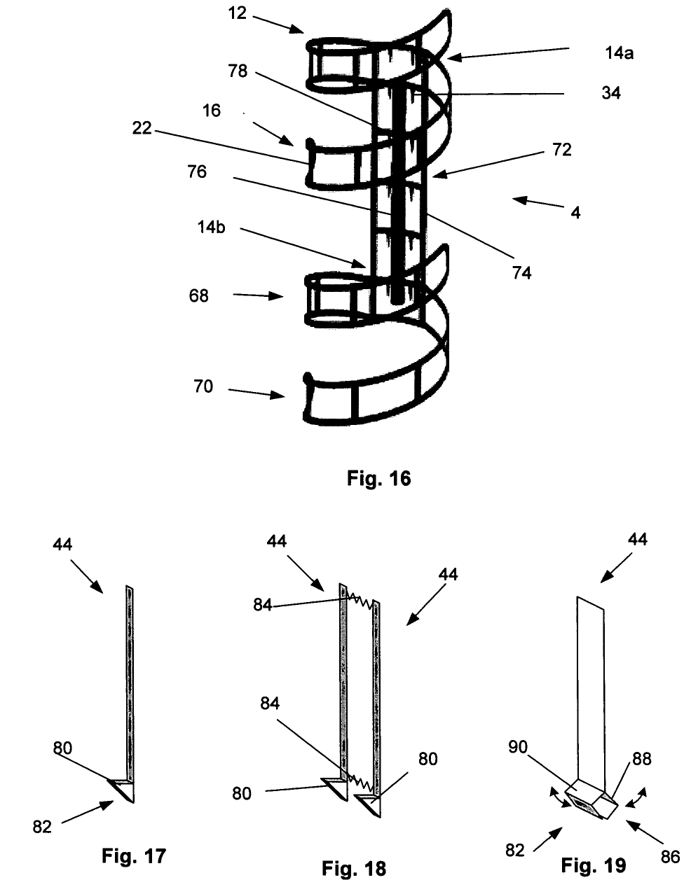

FIG. 16 illustrates a vascular fixation device 4 that can the first fixation section 14a that can be attached to the second fixation section 14b. The first arm 12 and the second arm 16 can extend from the first fixation section 14. A third arm 68 and a fourth arm 70 can extend from the second fixation section 14b.

A connecting brace 72 can fixedly or removably attach the first fixation section 14a to the second fixation section 14b. The connecting brace 72 can have side braces 74, a back brace 76 and cross braces 78. The cross braces 78 can attach one side brace 74 to another side brace 74 and/or one or both side braces 74 to the back brace 76. The back brace 76 can attach to the first and/or second connectors 36 and/or 38 on each fixation section 14a and 14b.

The terminus 22 of the second arm 16 can attach directly to the second fixation section 14 in lieu of the third arm 68 (not shown, also the terminus 22 previously on the third arm 68 could then no longer be a terminus 22). When the second arm 16 is directly attached to the second fixation section 14, the connecting brace 72 can be used or can be absent.

FIG. 17 illustrates the leg 44 that can have an interference member 80 at a distal end 82. FIG. 18 illustrates two legs 44 of FIG. 17 that can be attached to each other by resilient members 84. FIG. 19 illustrates the leg 44 that can have a crimp member 86 at the distal end 82. The crimp member 86 can have a first crimp side 88 and a second crimp side 90. The crimp sides 88 and 90 can be configured to resiliently angle outward from the leg 44, as shown by arrows.

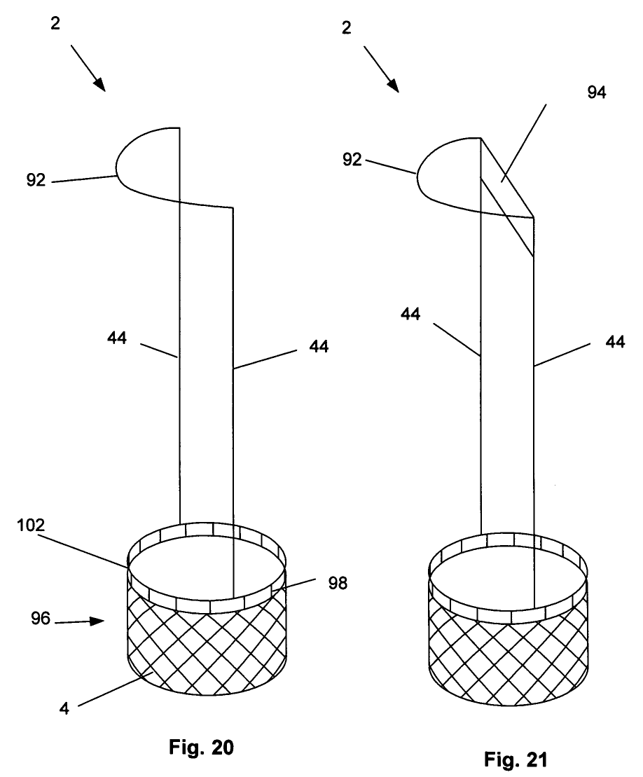

FIGS. 20 and 21 illustrate the intravascular graft anchoring assembly 2 that can have a first end 92 of the intravascular graft anchoring assembly 2. The first end 92 of the intravascular graft anchoring assembly 2 can be configured to fix to the vessel and can attach to the graft 8. The first end 92 can be substantially semicircular in shape. The first end 92 can be fixedly or resiliently attached to one or more legs 44. A back plate 94 can be attached to the first end 92 of the intravascular graft anchoring assembly 2 and/or the legs 44.

The legs 44 can be fixedly or resiliently attached to the graft attachment member 102 or 108 at the second end 96 of the intravascular graft anchoring assembly 2. The legs 44 can be resilient. The graft attachment member 102 or 108 can be attached to a suspension 98 that can effectively act as a mechanical spring and damper. The graft attachment member 102 or 108 can be attached directly to an expandable vascular fixation device 4. The vascular fixation device 4 can be a stent known to one having ordinary skill in the art, the vascular fixation devices 4 described infra and shown, for example, in FIGS. 1 through 16, or combinations thereof.

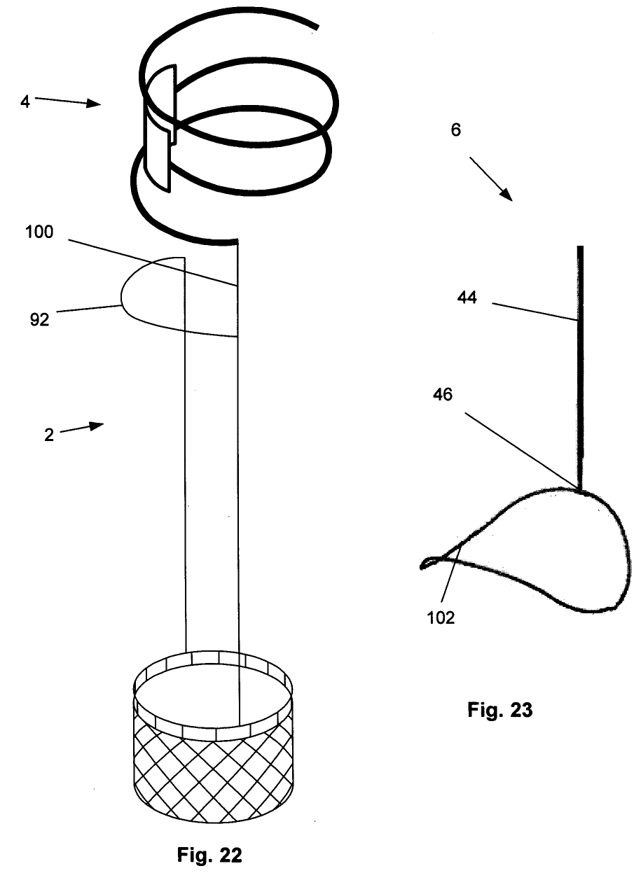

FIG. 22 illustrates the intravascular graft anchoring assembly 2 that can have the vascular fixation device 4 attached to the first end 92. The vascular fixation device 4 can be attached to the first end 92 by an extender 100.

FIG. 23 illustrates the graft attachment device 6. The graft attachment device 6 can have the leg 44. The leg 44 can attach to a first graft attachment member 102 at the leg attachment 46.

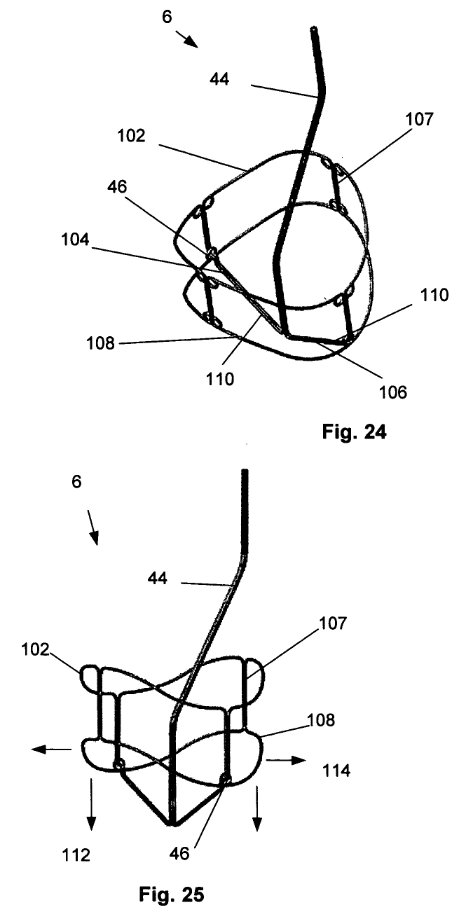

FIGS. 24 and 25 illustrate the graft attachment device 6 that can have a first section 104 and a second section 106. The leg attachments 46 can attach integrally or distinctly with the first graft attachment members 102, cross members 107, and second graft attachment members 108. The cross members 107 can integrally or distinctly attach the first graft attachment members 102 and the second graft attachment members 108. The graft 8 can fixedly or removably attach to the first graft attachment member 102, and/or the second graft attachment member 108, and/or the cross member 107 and/or the legs 44, for example, by crimping, snapping, sewing, stitching, gluing, welding, interference fitting (e.g., snapping), friction fitting and combinations thereof.

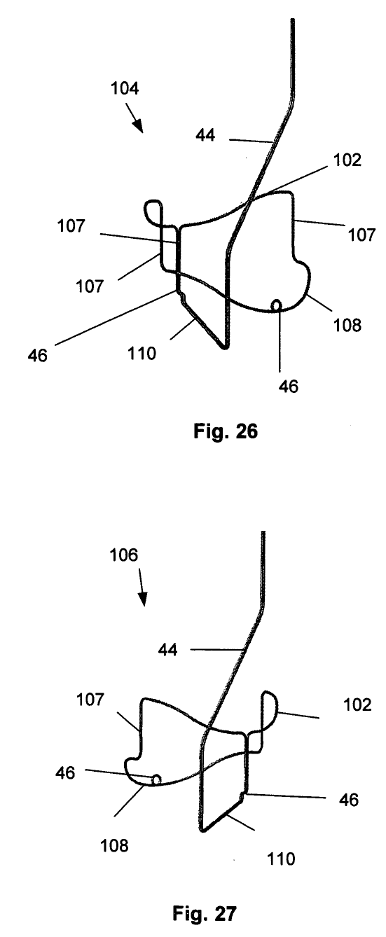

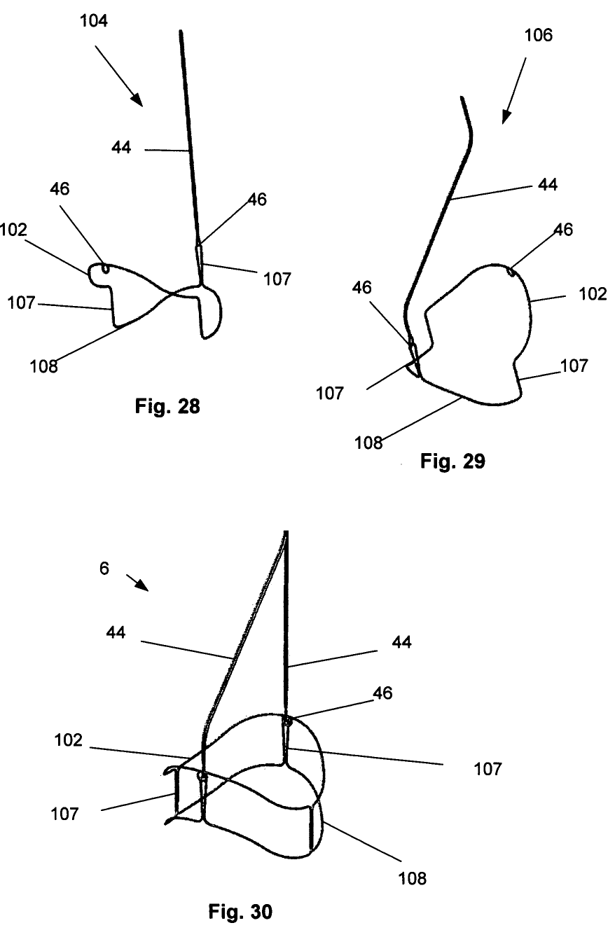

FIGS. 26 and 27 illustrate the first section 104 and the second section 106, respectively, of the graft attachment device 6 of FIGS. 24 and 25. FIGS. 28 and 29 illustrate the first section 104 and the second section 106 of the graft attachment device 6 that can have diverging legs 44 and is illustrated in FIG. 30.

The first graft attachment member 102 and the second graft attachment member 108 can have a scalloped shape (shown well in FIG. 23). The scalloped shape can facilitate a non-obstructing use of the graft attachment device 6 distal to vascular side branches off of the implantee vessel. Diverging legs 44 can have diverging branches 110. The diverging branches 110 can attach to the second end 42 of the graft attachment device 6 at the leg attachment 46. As shown in FIG. 25, when the graft attachment device 6 is exposed to a distally directed force, as shown by arrows 112, the graft attachment members 102 and/or 108 can radially expand or contract, as shown by arrows 114.

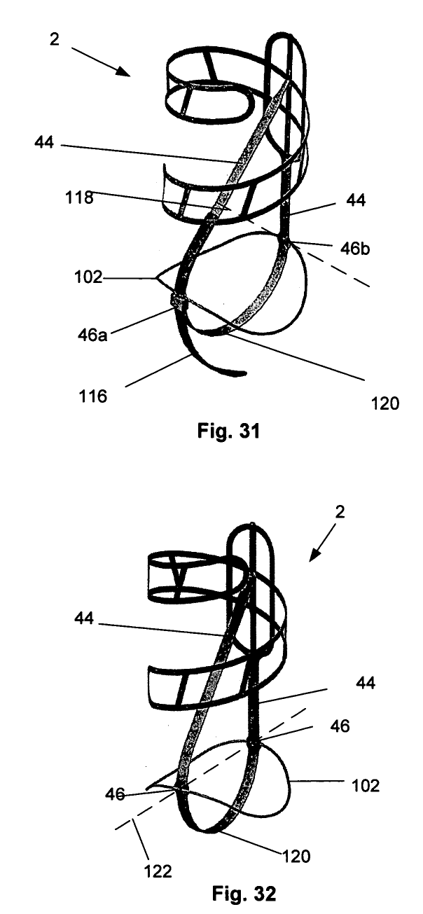

FIG. 31 illustrates an intravascular graft anchoring assembly 2 that can have a first graft attachment member 102 that can be fixedly attached to the first leg attachment 46a. A leg extension 116 can be fixedly attached to, and extend from, one of the legs 44. The first leg attachment 46a can be slidably attached to the leg extension 116. The first graft attachment member 102 can be rotatably attached to the second leg attachment 46b with respect to a first rotation axis 118. A converging branch 120 can attach one leg 44 to the other leg 44. FIG. 32 illustrates the intravascular graft anchoring assembly 2 that can have the first graft attachment member 102 that can be rotatably attached to the legs at the leg attachments 46 with respect to a second rotation axis 122.

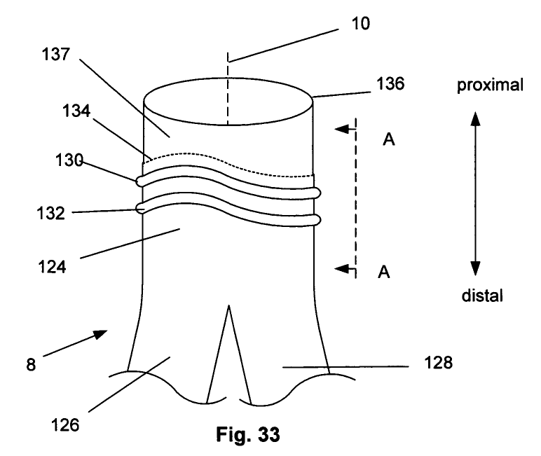

FIG. 33 illustrates the graft 8 that can have a graft body 124. The graft body 124 can be the graft trunk, or other entryway of flow through the graft 8). A first graft leg 126 and a second graft leg 128 can extend from the graft body 124. The graft body 124 can be fixedly attached to a first graft member 130 and a second graft member 132. The graft body 124 can have a reinforcement, described infra, that culminates at a reinforcement boundary 134 and/or a rim 136. The graft members 130 and 132 can be distinct members, a radially enlarged portion of the graft body 124, or combinations thereof. The graft 8 can have unreinforced graft 137 where the graft body 124 is not reinforced. The unreinforced graft 137 can be made from a polymer and/or metal weave made from a material described infra or combinations thereof.

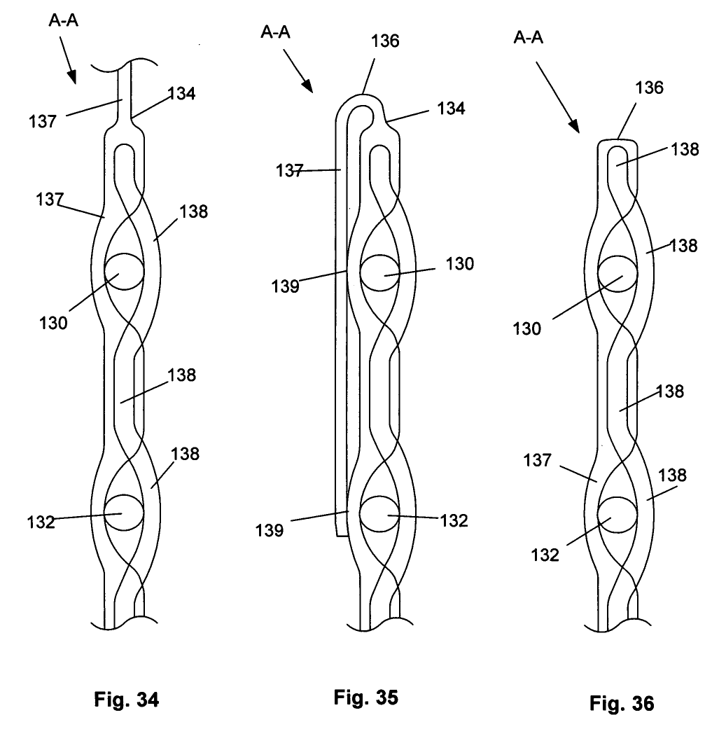

FIGS. 34 through 36 illustrates cross-section A-A of various grafts 8 that can have a reinforcement 138, for example a polymer and/or metal weave made from a material described herein or combinations thereof. FIG. 34 illustrates the graft 8 that can have the first graft member 130 and the second graft member 132 longitudinally separated. The first and second graft members 130 and 132 can be between the reinforcement 138 and the unreinforced graft 137. The reinforcement 138 can be disposed internally to the graft body 124 when not encapsulating the graft members 130 and 132. The portion of the unreinforced graft proximal to the reinforcement boundary can continue proximally until the rim 136.

FIG. 35 illustrates the graft 8 that can have the unreinforced graft 137 proximal to the reinforcement boundary 134 wrapped around the outside, or into the inside, of the graft body 124. The wrapped-around portion of the unreinforced graft 137 can be attached, for example by ultrasonic or heat welding, to the graft body 124 at wraparound fixation points 139. FIG. 36 illustrates the graft 8 that can have no reinforcement boundary 134. The reinforcement 138 can extend proximally to, or almost to, the rim 136.

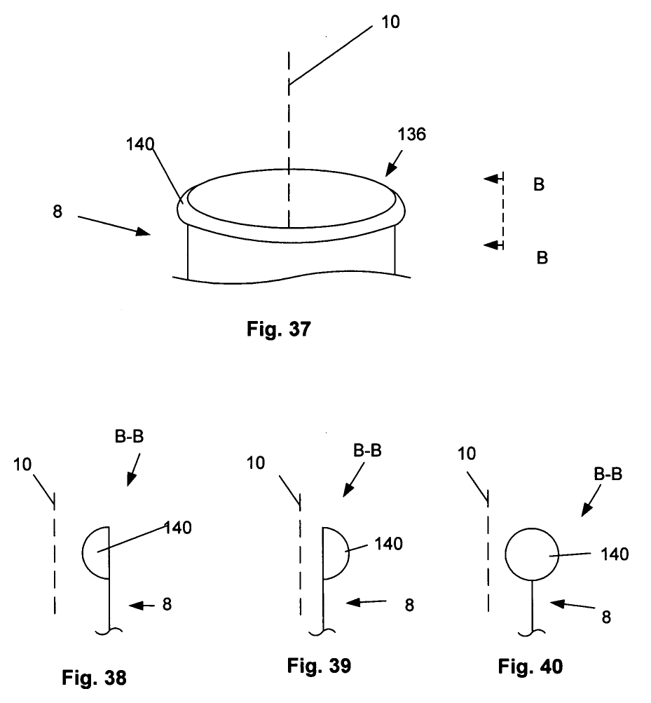

FIG. 37 illustrates the rim 136 that can have a lip 140. FIG. 38 illustrates the lip 140 that can extend radially inward toward the longitudinal axis 10. FIG. 39 illustrates the lip 140 that can extend radially outward away from the longitudinal axis 10. FIG. 40 illustrates the lip 140 that can extend proximally and/or radially inward and radially outward with respect to the longitudinal axis 10.

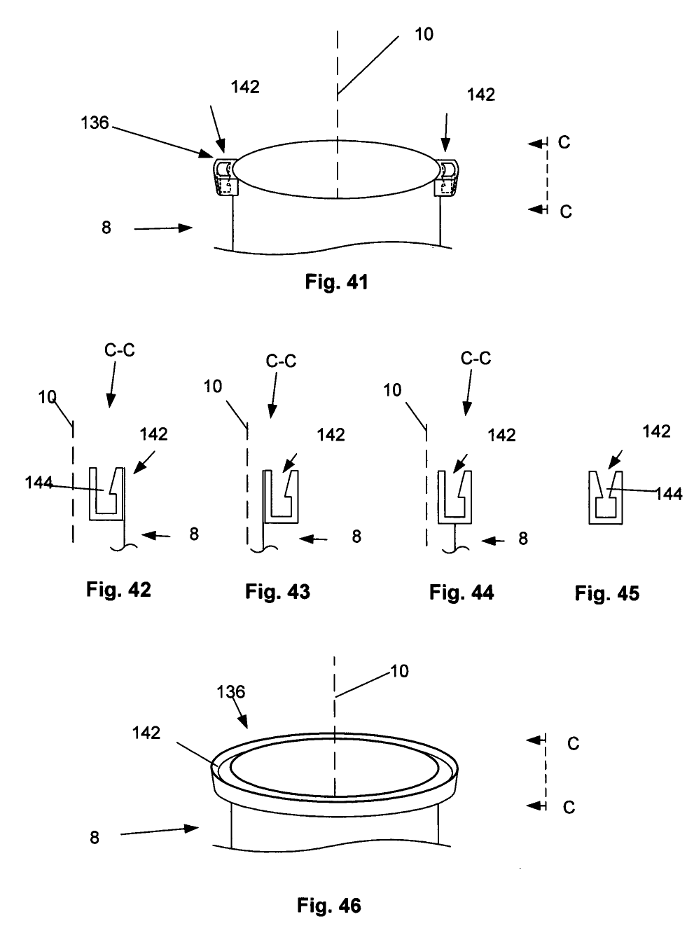

FIG. 41 illustrates the rim 136 that can have one or more interference receptacles 142. FIG. 42 illustrates that the interference receptacle 142 can have, for example, a unilateral snap-lock port. The interference receptacle 142 can extend radially inward toward the longitudinal axis 10. FIG. 43 illustrates the interference receptacle 142 that can extend radially outward away from the longitudinal axis 10. FIG. 44 illustrates the interference receptacle 142 that can extend proximally and/or radially inward and radially outward with respect to the longitudinal axis 10. FIG. 45 illustrates a cross-section of the interference receptacle 142 that can have, for example, a bilateral snap-lock port 144.

FIG. 46 illustrates the rim 136 that can have the interference receptacle 142 that can circumferentially cover the rim 136. The cross-sections illustrated in FIGS. 42 through 45 can be for the graft 8 of FIG. 46.

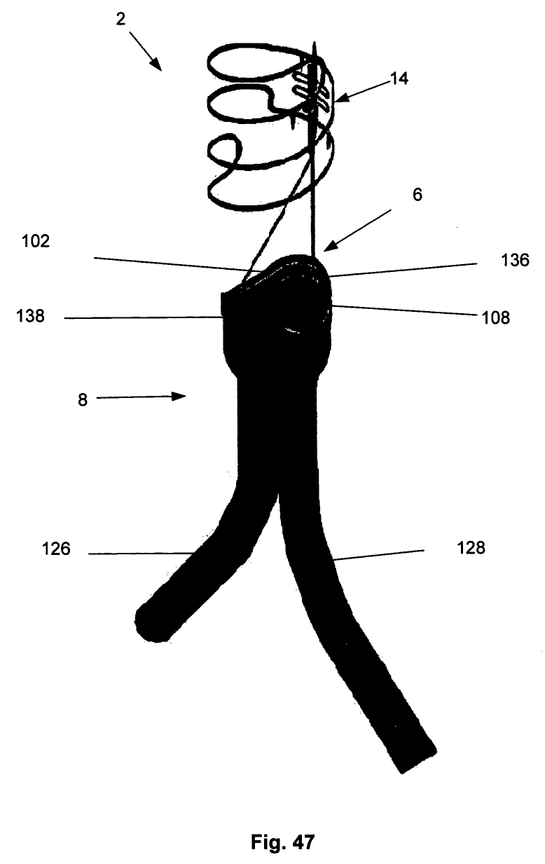

FIG. 47 illustrates the intravascular graft anchoring assembly 2 attached to the graft 8. The first and second graft attachment members 102 and 108 can interference fit with the first and second graft members 130 and 132 (not shown). The graft 8 can have bifurcating graft legs 126 and 128. The reinforcement 138 can provide sufficient radial support to keep the rim 136 open without additional radial force from the graft attachment device 6.

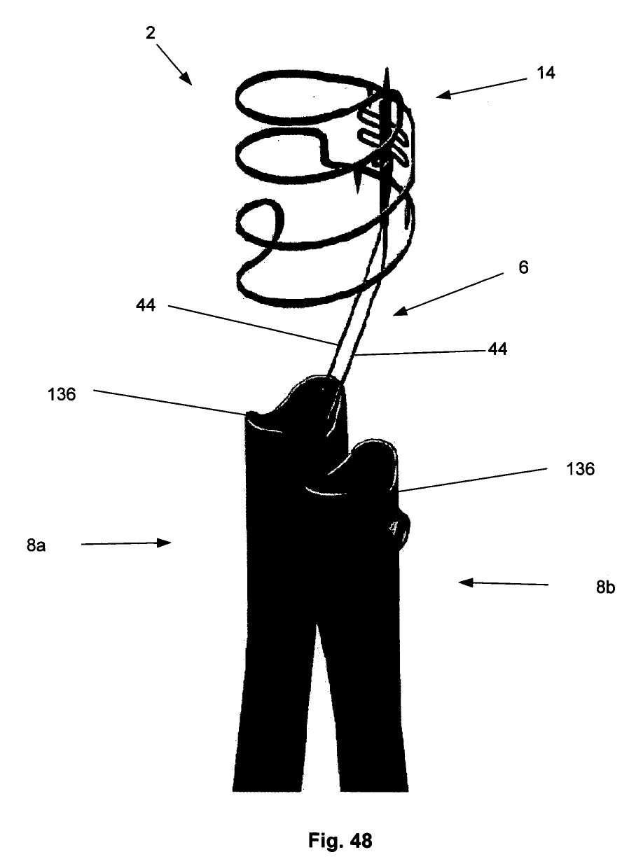

FIG. 48 illustrates the intravascular graft anchoring assembly 2 attached to the first graft 8a and the second graft 8b. The legs 44 can be attached directly to the grafts 8a and 8b. The legs 44 can attach to second ends 42 of two graft attachment devices 6 (not shown). The second ends 42 of the two graft attachment devices 6 can separately attach to their respective graft 8a or 8b.

Methods of Manufacture

The elements of the intravascular graft anchoring assembly 2 can be directly attached by, for example, melting, screwing, gluing, welding or use of an interference fit or pressure fit such as crimping, or combining methods thereof. The elements can be integrated, for example, molding, die cutting, laser cutting, electrical discharge machining (EDM) or stamping from a single piece or material. Any other methods can be used as known to those having ordinary skill in the art.

Integrated parts can be made from pre-formed resilient materials, for example resilient alloys (e.g., Nitinol, ELGILOY®) that are preformed and biased into the post-deployment shape and then compressed into the deployment shape as known to those having ordinary skill in the art.

Any elements of the intravascular graft anchoring assembly 2, or the intravascular graft anchoring assembly 2 as a whole after assembly, can be coated by dip-coating or spray-coating methods known to one having ordinary skill in the art. One example of a method used to coat a medical device for vascular use is provided in U.S. Pat. No. 6,358,556 by Ding et al. and hereby incorporated by reference in its entirety. Time release coating methods known to one having ordinary skill in the art can also be used to delay the release of an agent in the coating. The coatings can be thrombogenic or anti-thrombogenic. For example, coatings on the inside of the intravascular graft anchoring assembly 2, the side facing the longitudinal axis 10 can be anti-thrombogenic, and coatings on the outside of the intravascular graft anchoring assembly 2, the side facing away from the longitudinal axis 10, can be thrombogenic.

The intravascular graft anchoring assembly 2 can be covered with a fabric, for example polyester (e.g., DACRON® from E. I. du Pont de Nemours and Company, Wilmington, Del.), polypropylene, PTFE, ePTFE, nylon, extruded collagen, silicone or combinations thereof. Methods of covering an implantable device with fabric are known to those having ordinary skill in the art.

Method of Using

The intravascular graft anchoring assembly 2 can be radially collapsed and loaded into one or more delivery sheaths or catheters 146, as known to one having ordinary skill in the art. The graft 8 can be attached to the intravascular graft anchoring assembly 2 before being collapsed and loaded into the delivery catheter 146, or via a separate delivery catheter after the intravascular graft anchoring assembly 2 is deployed.

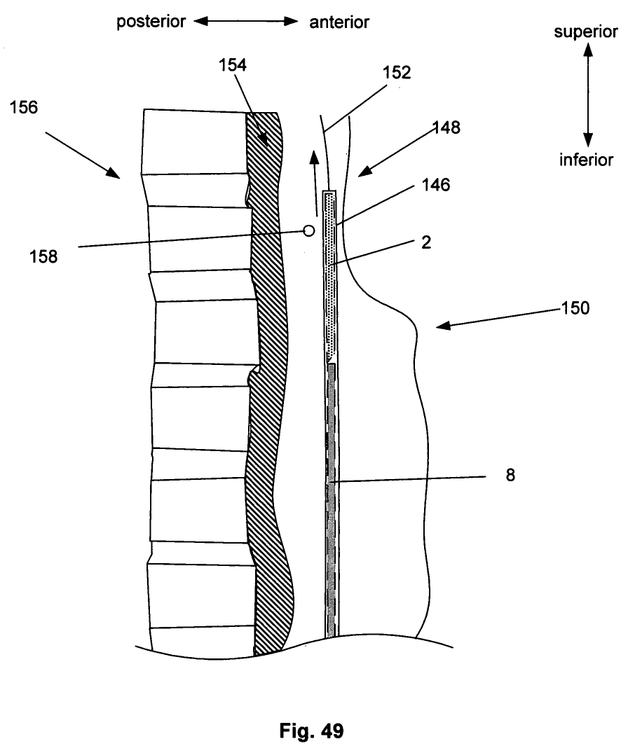

FIGS. 49 through 51 illustrate a method of deploying the intravascular graft anchoring assembly 2 into a vascular site 148, for example proximal to an abdominal or thoracic aortic aneurysm 150, with one or more delivery catheters 146. After a guidewire 152 is deployed to the vascular site 148, the delivery catheter 146 can be moved along the guidewire 152 until the intravascular graft anchoring assembly 2 is in position to be expanded.

The vascular site 148 can have a portion of wall that is substantially fixed with respect to the remainder of the wall of the vascular site 148. For example, the posterior portion of the vascular site 148 shown in FIGS. 49 through 51 is substantially fixed in place by connective tissue 154 that fixes the vascular site 148 to the spine 156. The delivery catheter 146 can be oriented so the fixation section 14 can be deployed adjacent to the substantially fixed portion of the vascular site 148, for example the portion closest to the connective tissue 154.

The intravascular graft anchoring assembly 2 can be positioned prior to deployment so that the vascular fixation device 4 can be deployed superior to lateral vessel branches, for example the orifice for the renal artery 158. The intravascular graft anchoring assembly 2 can be positioned prior to deployment so that the second end of the graft attachment device 6 can be deployed inferior to lateral vessel branches, for example the orifice for the renal artery 158.

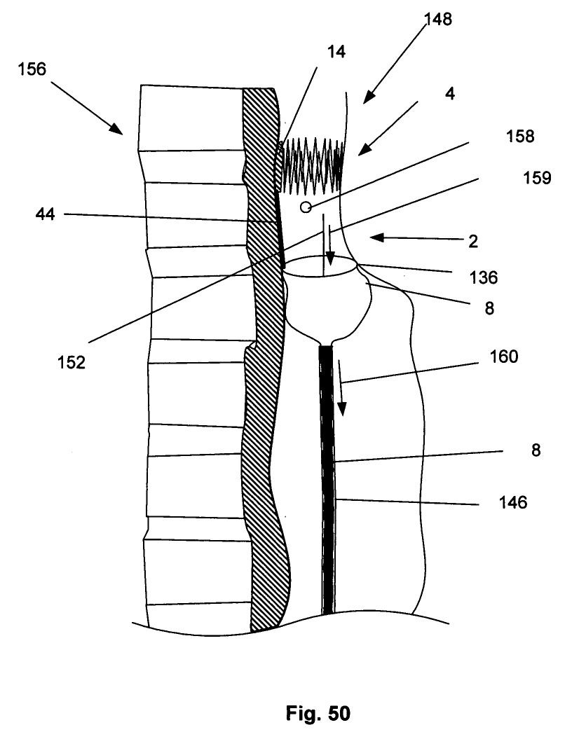

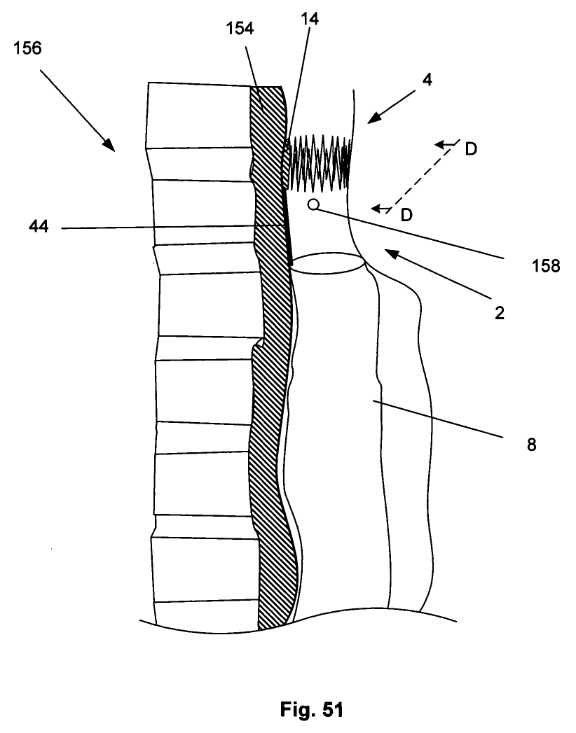

As FIG. 50 illustrates, the guidewire can be withdrawn as shown by arrow 159. The catheter 146 can be withdrawn, as shown by arrow 160. When the catheter 146 is withdrawn, as shown by arrow 160, the intravascular graft anchoring assembly 2 can be deployed at the vascular site 148 with the fixation section 14 superior to the renal artery 158 and the second end 42 (not shown) of the graft attachment device 6 (or the rim 136 of the graft 8 when the second end 42 of the graft attachment device 6 is not present), inferior to the renal artery 158. The fixation section 14 can be deployed adjacent to the spine 156. FIG. 51 illustrates the fully deployed intravascular graft anchoring assembly 2 attached to the fully deployed graft 8 with the delivery catheter 146 and guidewire 152 removed from the vascular site 148 and the aneurysm 150.

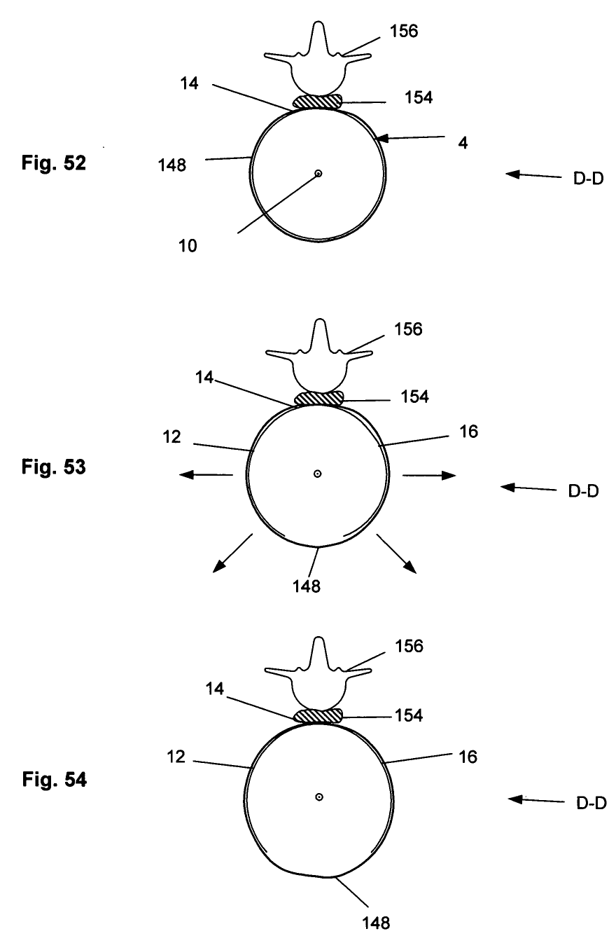

FIG. 52 illustrates cross-section D-D at diastole. With the vascular site 148 fully contracted, the first and second arms 12 and 16 (not distinctly shown) can be in a fully contracted configuration to fit the vascular site 148.

FIG. 53 illustrates cross-section D-D after diastole and before systole. As the vascular site 148 naturally expands circumferentially, as shown by arrows, away from the connective tissue 154, the fixation section 14 can stay fixed to the vascular site 148 adjacent to the connective tissue 154 and the first and second arms 12 and 16 can expand to fit the expanding vascular site 148.

FIG. 54 illustrates cross-section D-D at systole. With the vascular site 148 fully dilated and expansion of the vascular site 148 having stopped, the first and second arms 12 and 16 can be in an expanded configuration to fit the vascular site 148. The fixation section 14 can remain fixed to the vascular site 148 adjacent to the connective tissue 154.

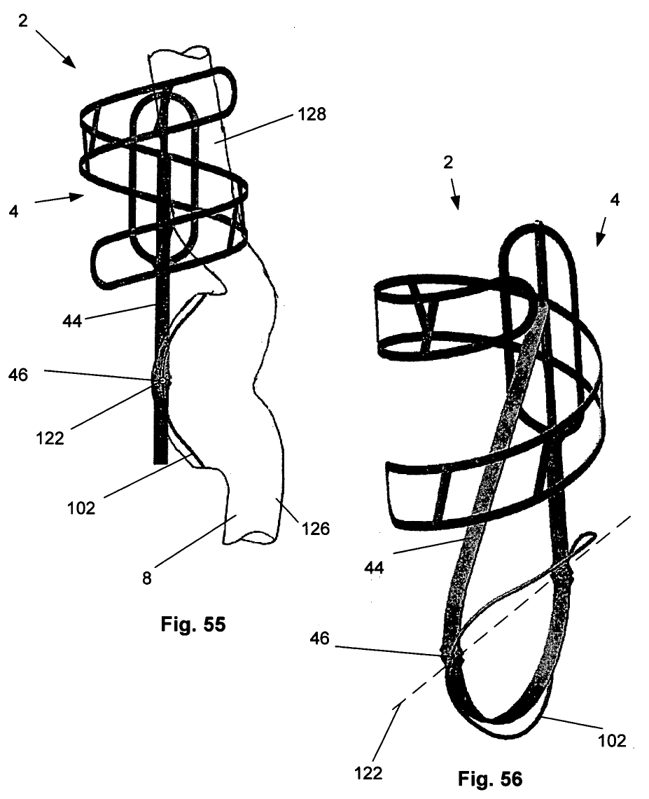

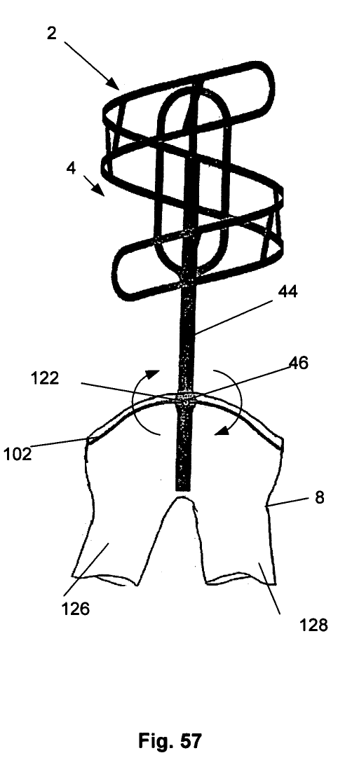

FIGS. 55 and 56 illustrate a method of deploying the graft 8 using an intravascular graft anchoring assembly 2 that can have the second rotational axis 122, similar to that of the intravascular graft anchoring assembly 2 of FIG. 32. FIG. 55 illustrates the graft 8 in a collapsed configuration. The first graft leg 126 can be fed into or adjacent to the vascular fixation device 4 to reduce the deployment cross-section. The second graft leg 128 can be placed distal to the intravascular graft anchoring assembly 2. FIG. 56 illustrates the intravascular graft anchoring assembly 2 of FIG. 32 in a collapsed configuration without the graft 8. FIG. 57 illustrates that upon deployment, the first graft attachment member 102, and therefore the graft 8, can be rotated, as shown by arrows, with respect to the second rotational axis 122 into an expanded, deployed configuration.

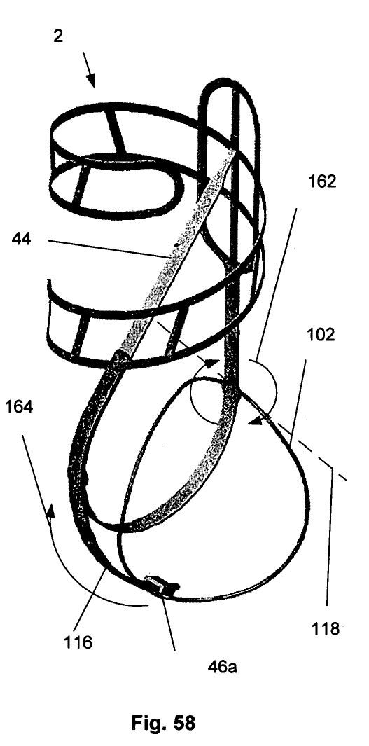

FIG. 58 illustrates the intravascular graft anchoring assembly 2 of FIG. 31 in a collapsed configuration. The first graft attachment member 102 can be rotated, as shown by arrows 162, with respect to the first rotational axis 118. The leg attachment 46 can slide, as shown by arrow 164, along the leg extension 116. Upon deployment, the first graft attachment member 102 can be rotated with respect to the first rotational axis 118 into an expanded, deployed configuration, as shown in FIG. 31.

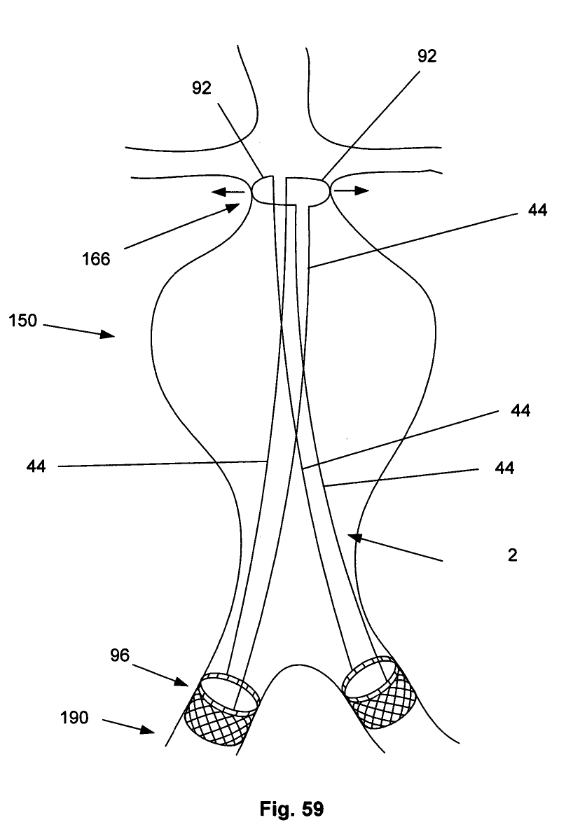

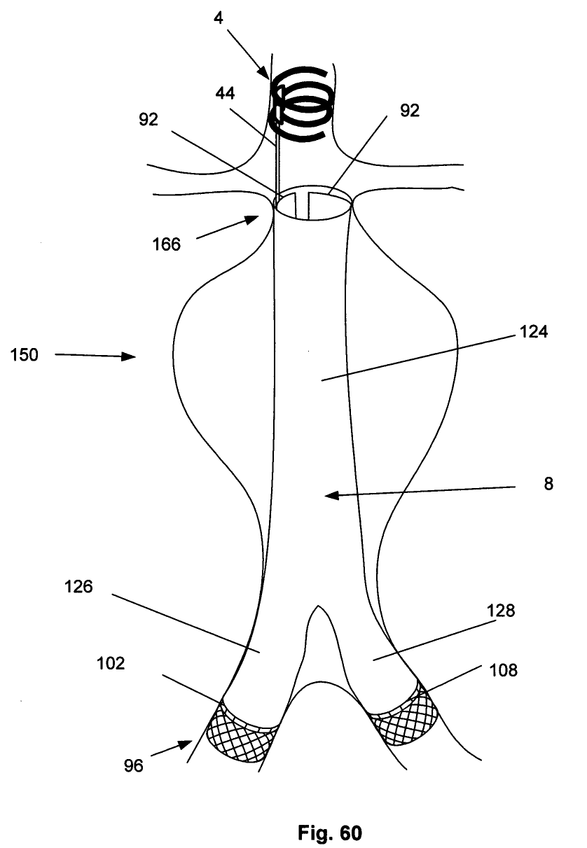

FIGS. 59 illustrates deploying the intravascular graft anchoring assembly 2 of FIG. 20 in a vessel, for example across the aneurysm 150. The first end 92 of one or more intravascular graft anchoring assemblies 2 can be deployed to a neck 166 of the aneurysm 150. The legs 44 can be of a selected length such that the second end 96 of the intravascular graft anchoring assembly 2 can be deployed on an opposite side of the aneurysm 150 from the first end 92 of the intravascular graft anchoring assembly 2. For example, the second end 96 of the intravascular graft anchoring assembly 2 can be deployed in the iliac arteries 190 and 192 for an abdominal aneurysm 150. The resiliently deformed legs 44 can apply a force, shown by arrows, fixing the first ends 92 of the intravascular graft anchoring assemblies 2 against the neck 166.

FIG. 60 illustrates the graft 8 deployed on the intravascular graft anchoring assemblies 2 of FIG. 22. One end of the graft 8 can be attached to the first ends 92 the intravascular graft anchoring assemblies 2. The other ends of the graft 8 can be attached to the graft attachment members 102 and 108 at the second ends 96 of the intravascular graft anchoring assemblies 2.

One intravascular graft anchoring assembly 2 can be deployed followed by the deployment of the graft body 124 on the first end 92 of the deployed intravascular graft anchoring assembly 2. The graft body 124 can be attached to the first end 92 of the deployed intravascular graft anchoring assembly 2. A second intravascular graft anchoring assembly 2 can then be deployed so that the first end 92 of the newly deployed intravascular graft anchoring assembly 2 can attach to the graft body 124 adjacent to the first end 92 of the already-deployed intravascular graft anchoring assembly 2. Graft legs 44 can then be deployed over the intravascular graft anchoring assemblies 2. The graft legs 44 can be attached to the graft body 124 and to the graft attachment members 102 and 108 on the second ends 96 of the intravascular graft anchoring assemblies 2.

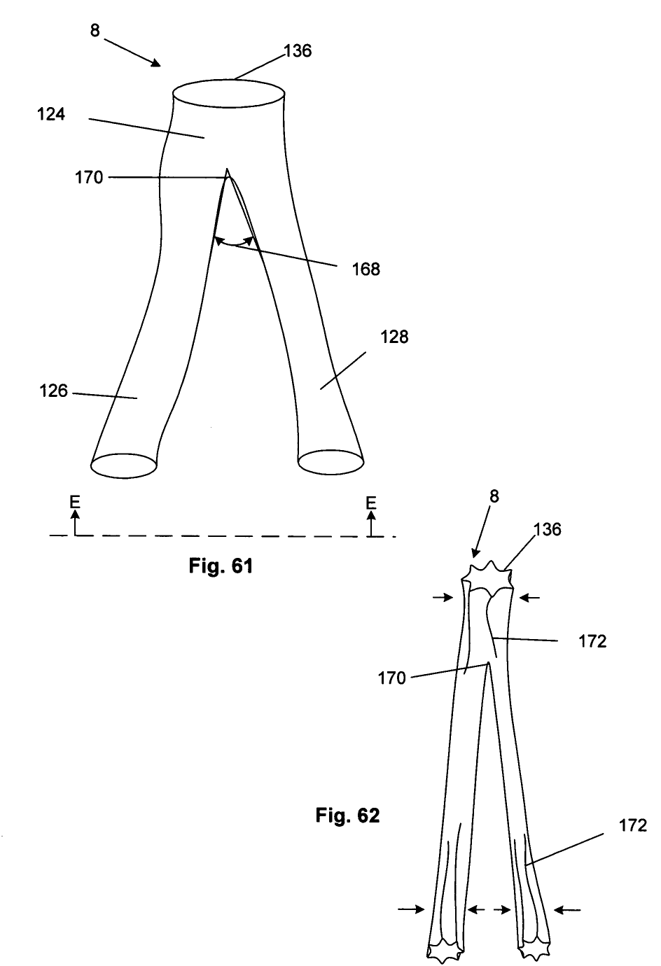

FIG. 61 illustrates the graft 8 that can have a bifurcation angle 168. The bifurcation angle can be the angle from the first graft leg 126 to the second graft leg 128. The bifurcation angle 168 can vary during use. The bifurcation angle 168 can be from about 0° to about 360°, for example about 30°. The graft body 124 can have a septum 170. The septum can separate the first graft leg 126 and the second graft leg 128.

FIG. 62 illustrates a method of compressing the graft 8 to prepare the graft 8 for deployment, for example minimally invasive deployment. Radially compressive forces, as shown by arrows, can radially compress the graft 8 and the intravascular graft anchoring assembly 2 (not shown) as illustrated by compression folds 172.

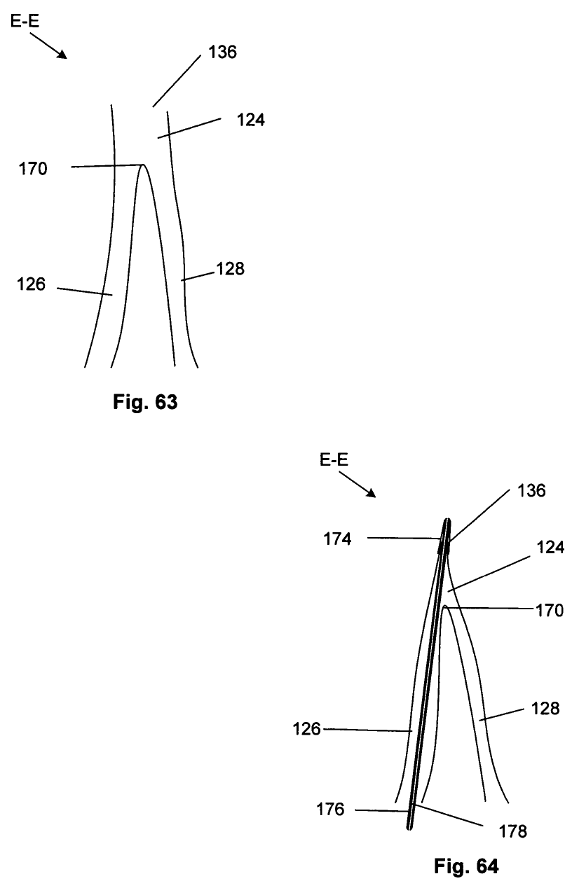

FIG. 63 illustrates cross-section E-E of FIG. 61. FIG. 64 illustrates attaching the rim 136 of the graft 8 to a temporary fixator 174 on a temporary fixator shaft 176. The graft 8 can be attached to the intravascular graft anchoring assembly 2 (not shown, but can be attached to the graft 8 in FIGS. 64-77). The temporary fixator shaft 176 can be placed in the first graft leg 126 and the graft body 124. The temporary fixator shaft 176 can have a lumen 178, for example a lumen for passing the guidewire 152 therethrough. The temporary fixator 174 can be an adhesive, an interference fit (e.g., a snap), a friction fit (e.g., a bell) or combinations thereof.

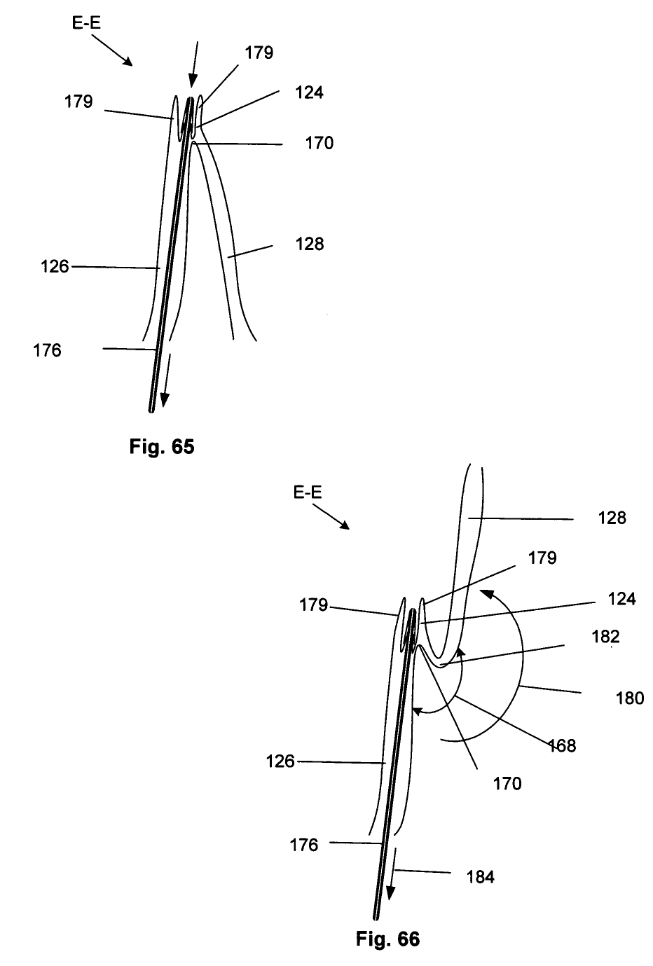

FIG. 65 illustrates invaginating the rim 136 into the graft body 124. The rim 136 can be left in a non-invaginated configuration during deployment. The temporary fixator shaft 176 can be pulled, as shown by arrows. As the rim 136 invaginates into the graft body 124, one or more inversion folds 179 can form around the rim 136.

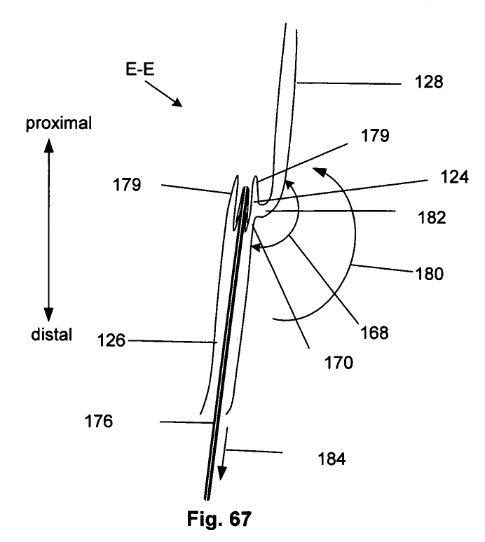

FIGS. 66 and 67 illustrate folding, as shown by arrow 180, the second graft leg 128 into a pre-deployment configuration. The second graft leg 128 can be folded at a fold point 182. The fold point 182 can be located away from the septum 170, as shown in FIG. 66. The fold point 182 can be located near or on the septum 170, as shown in FIG. 67. The rim 136 can be further invaginated into the graft body 124 and/or first graft leg 126, as shown by arrow 184. In a pre-deployment configuration, the bifurcation angle 168 can be from about 90° to about 270°, more narrowly from about 120° to about 250°, yet more narrowly from about 165° to about 195°, for example about 180°.

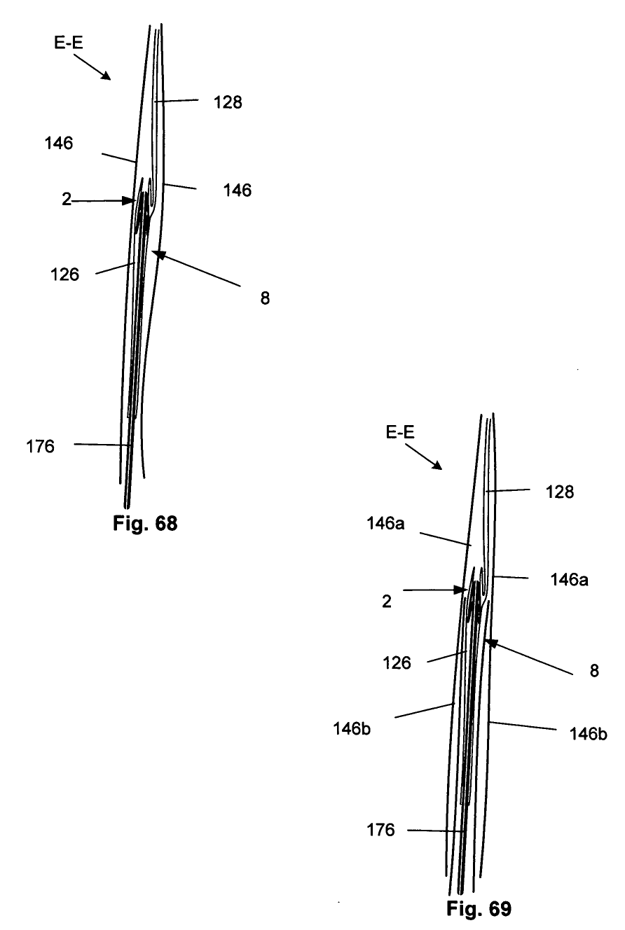

FIG. 68 illustrates the graft 8 compressed, as shown in FIG. 62, and inserted into the delivery catheter 146. The inside and/or outside of the delivery catheter 146 can be coated with lubricious and/or therapeutic materials and/or agents.

FIG. 69 illustrates the graft 8 compressed and inserted into the first delivery catheter 146a and the second delivery catheter 146b. The first delivery catheter 146a can be temporarily attached to the second delivery catheter 146b. The first delivery catheter 146a can cover the entire graft 8. The first delivery catheter 146a can only cover enough of the graft 8 so as to attach the first delivery catheter 146a to the second delivery catheter 146b. The second delivery catheter 146b can extend from beyond the first graft leg 126. The second delivery catheter 146b can cover the graft 8 up to the inversion fold 179.

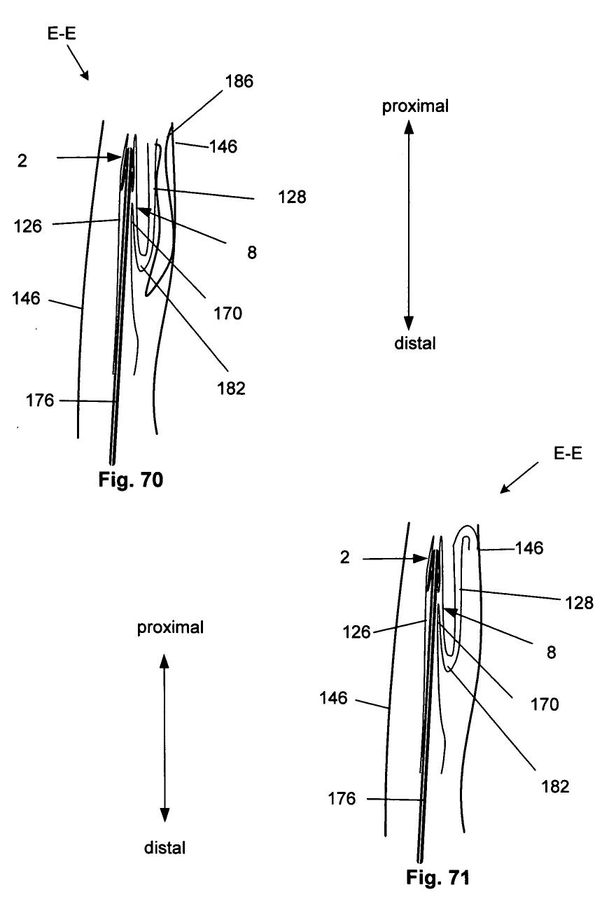

FIG. 70 illustrates the graft 8 compressed and inserted into the delivery catheter 146. (For clarity, the delivery catheter 146 is illustrated spaced away from the graft 8 in FIGS. 70 and 71.) The fold point 182 can be located anywhere along the septum or the second graft leg 128. The proximal end of the folded second graft leg 128 can be removably attached to a first end of a tether 186. A second end of the tether 186 can be removably attached to the inside, outside or any combination thereof, of the delivery catheter 146. When assembled as shown in FIG. 70, the tether 186 can have slack length.

FIG. 71 illustrates the graft 8 compressed and inserted into the delivery catheter 146. The proximal end of the already-folded second graft leg 128 can be folded again, so the open end of the folded second graft leg 128 is directed in a distal direction. The proximal end of the twice-folded second graft leg 128 can be removably attached to the inside, outside or any combination thereof, of the delivery catheter 146.

The intravascular graft anchoring assembly 2 can be attached to the proximal end of the graft body 124 prior to, or during, deployment. The intravascular graft anchoring assembly 2 can be compressed with the graft body 124. The intravascular graft anchoring assembly 2 can be placed in the delivery catheter 146 with the graft body 124. The preparation for deployment can be part of the deployment, itself.

FIGS. 72-84 illustrate methods of deploying the graft 8 and/or the intravascular graft anchoring assembly 2 in a patient, for example to treat an aortic aneurysm, such as a thoracic or abdominal aortic aneurysm. FIG. 72 illustrates the aortic aneurysm 150, part of the suprarenal aorta 188, the first and second iliac arteries 190 and 192, the internal iliac (i.e., hypogastric) arteries 194, and the renal arteries 196, all in cross-section.

Vascular access devices 197 can be inserted into the patient's blood system, for example, into the femoral or iliac arteries 190 and 192. The guidewire 152 can be fed through the vascular access devices 197, across the first iliac artery 190 and the second iliac artery 192, as shown by the arrow in FIG. 72. A snare (not shown), as known to one having ordinary skill in the art, can be used to steer the guidewire 152, for example, to pull it into the second iliac artery 192.

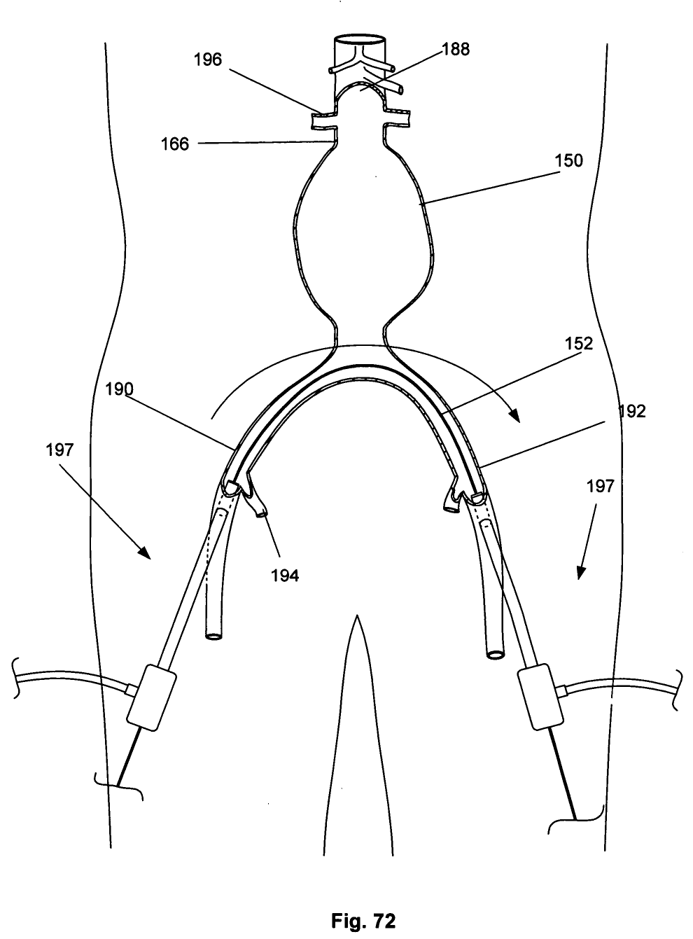

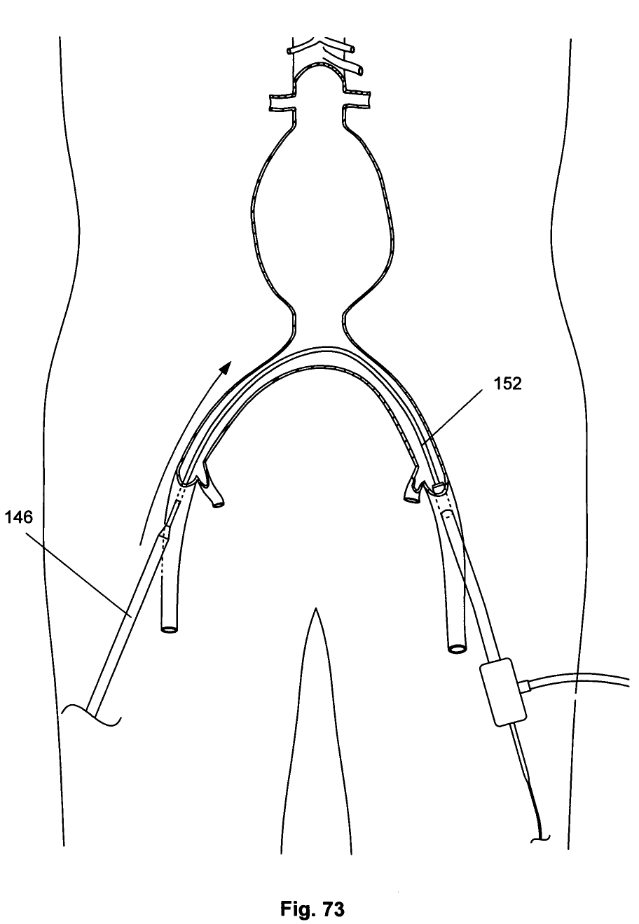

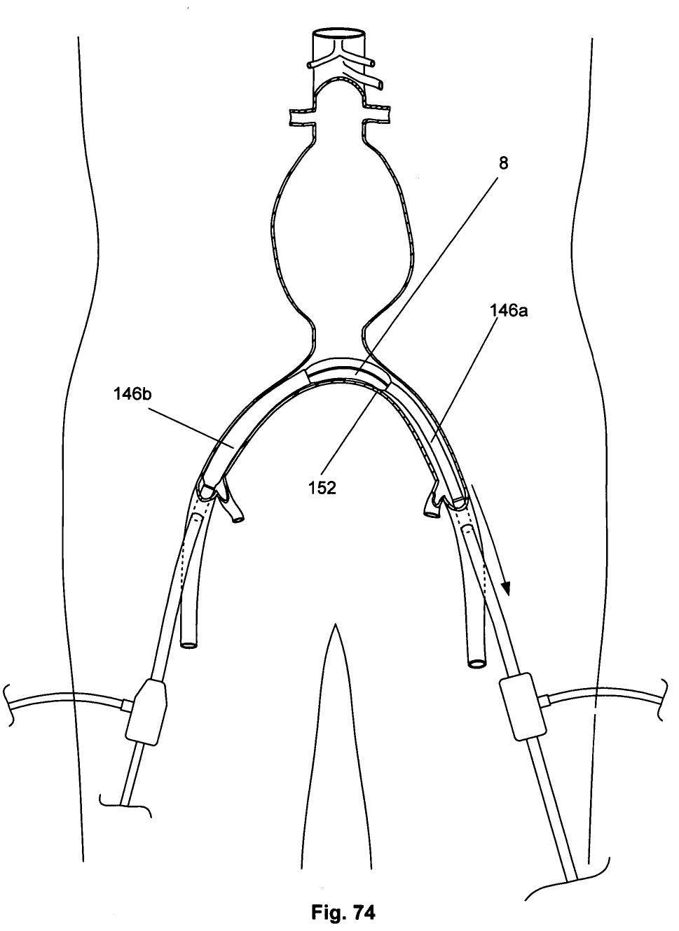

The guidewire 152 can be fed through the lumen 178 in the temporary fixator shaft 176. The graft 8, for example in a collapsed configuration and perhaps surrounded by the delivery catheter 146, can be deployed, as shown by the arrow in FIG. 73, over the guidewire 152.

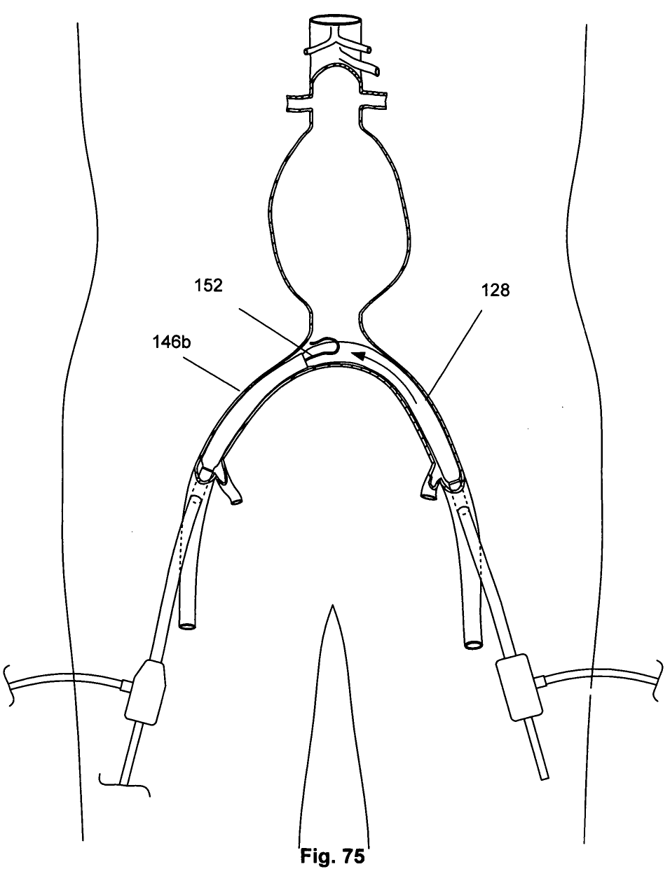

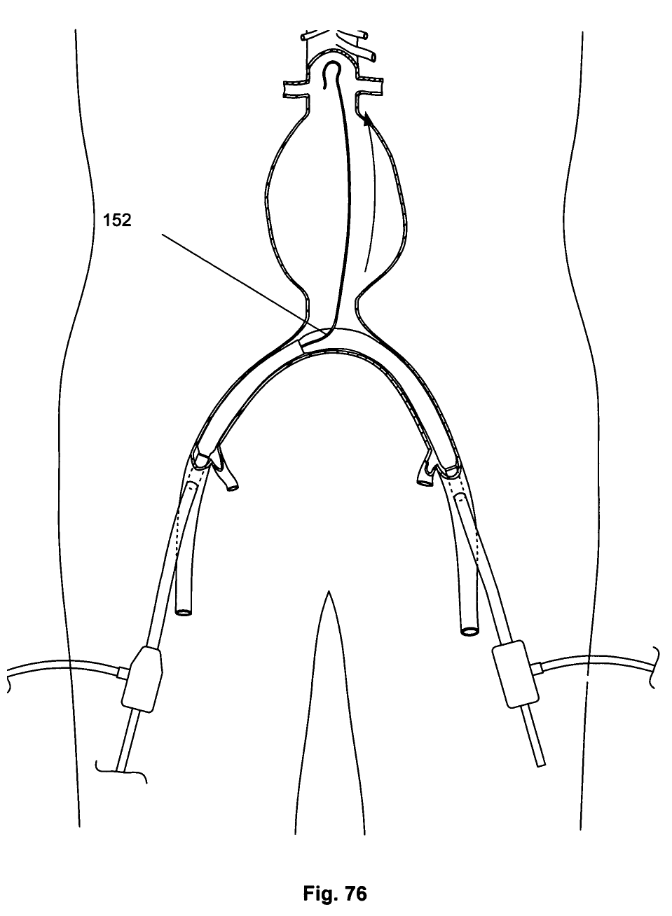

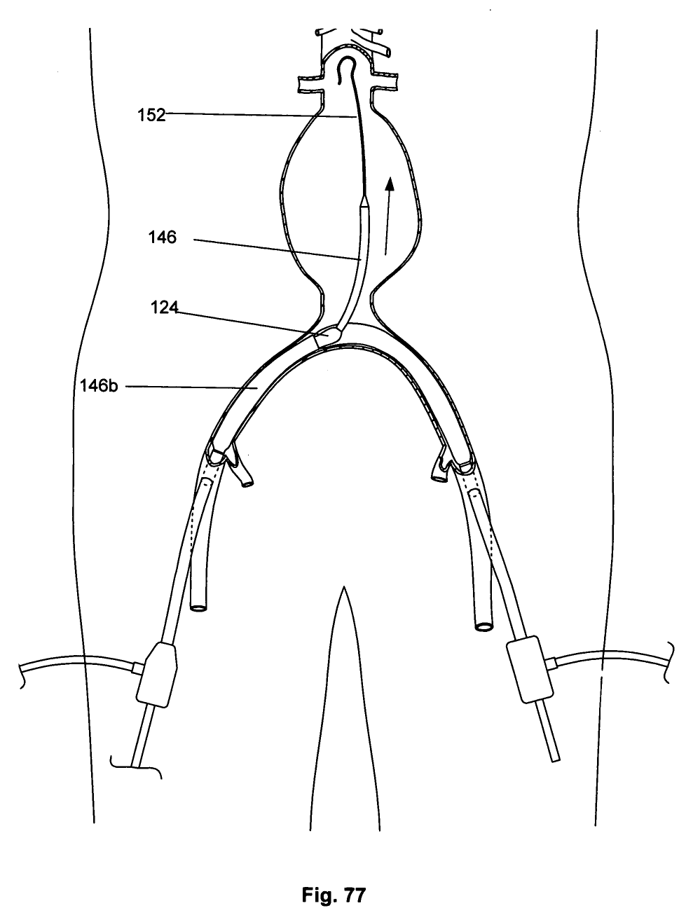

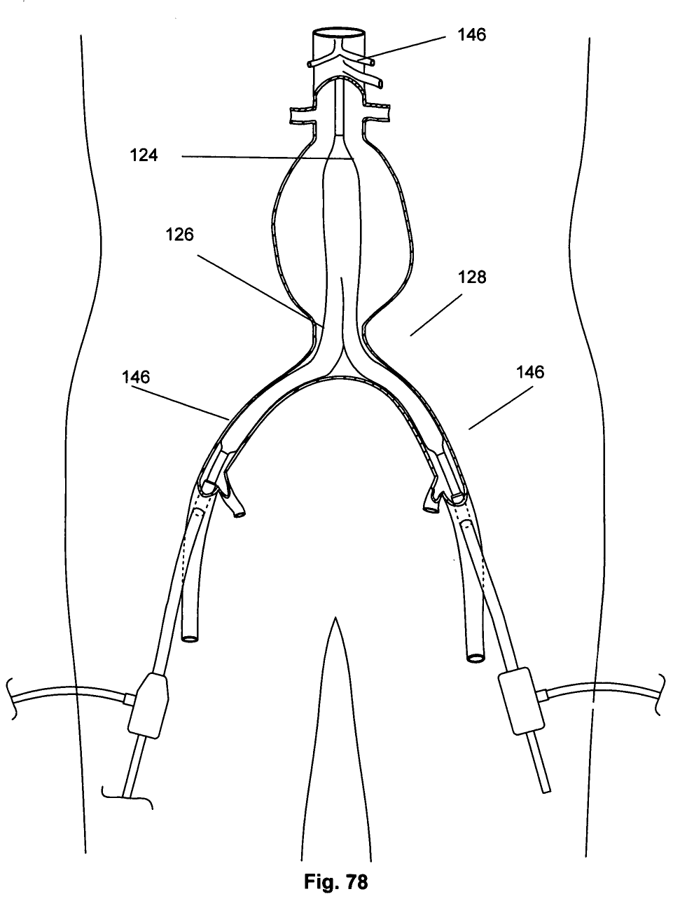

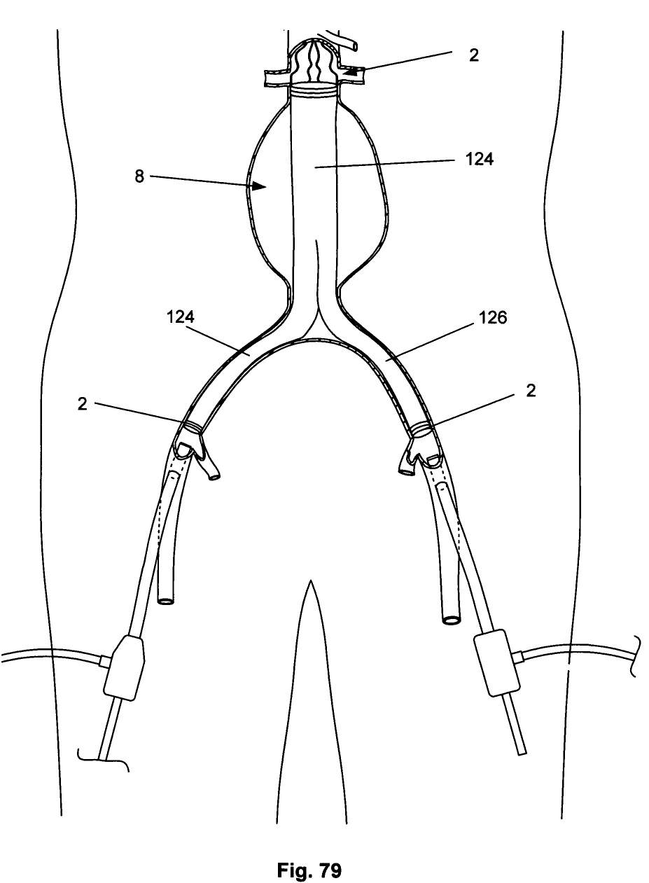

After the graft 8 is completely deployed in the iliac arteries 190 and 192, the first delivery catheter 146 can be removed from the graft. The second graft leg 128 can deploy into the second iliac artery 192. The guidewire 152 can be pulled back, as shown by the arrow in FIG. 75, toward the first iliac artery 190 so that the end of the guidewire 152 is near, and can access, the aneurysm 150.

The guidewire 152 can be deployed across the aneurysm and into the suprarenal aorta 188, as shown by arrow in FIG. 76. In FIG. 77, the graft body 124 (and the intravascular graft anchoring assembly 2 that can still be in a delivery catheter 146) can be deployed over the guidewire 152. The second delivery catheter 146 (or the remainder of the first delivery catheter 146) can be removed from the graft 8, as shown by FIG. 78. The first graft leg 126 can deploy into the first iliac artery 190. Graft leg end delivery catheters 146 can be over the ends of the graft legs 126 and 128.

FIG. 79 illustrates that the intravascular graft anchoring assembly 2 can be deployed, for example, in and near the suprarenal aorta 188. The intravascular graft anchoring assembly 2 can be attached to the graft 8. The length of the first and second graft legs 126 and 128 can be cut to a desired size, for example so as not to minimize impairment of the flow of the internal iliac arteries 194. Once the graft legs 126 and 128 are initially deployed in the vessel, for example, in the iliac arteries 190 and 192, the ends of the graft legs 126 and 128 can be cut, for example, by an intravascular or transvascular severing device. Examples of intravascular and transvascular severing devices include those as disclosed in U.S. Pat. Nos. 6,328,749 and 5,843,102 both to Kalmann et al., which are herein incorporated by reference in their entireties. Some transvascular severing devices can be scaled down to permit use as an intravascular severing device. The graft legs 126 and 128 can be cut by extending the ends of the graft legs 126 and 128 to extend the ends of the graft legs 126 and 128 into the vascular access devices 197 and/or out of the body entirely, to gain sufficient access to cut the graft legs 126 and 128 to a desired length with, for example, a suture or scissors. Energy can be transmitted (e.g., electrical current, RF radiation, heat) to the graft legs 126 and 128 to cut or assist cutting.

Excess material remaining on the graft legs 126 and 128 can then be corrugated into or near the iliac arteries 190 and 192. Intravascular graft anchoring assemblies 2 can be deployed at the ends of the graft legs 126 and 128. Other expandable vascular prostheses, for example stents, can be deployed at the ends of the graft legs 126 and 128.

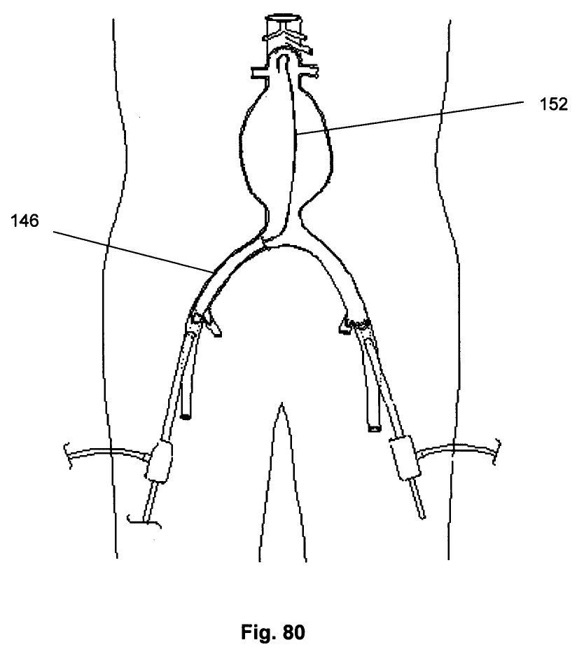

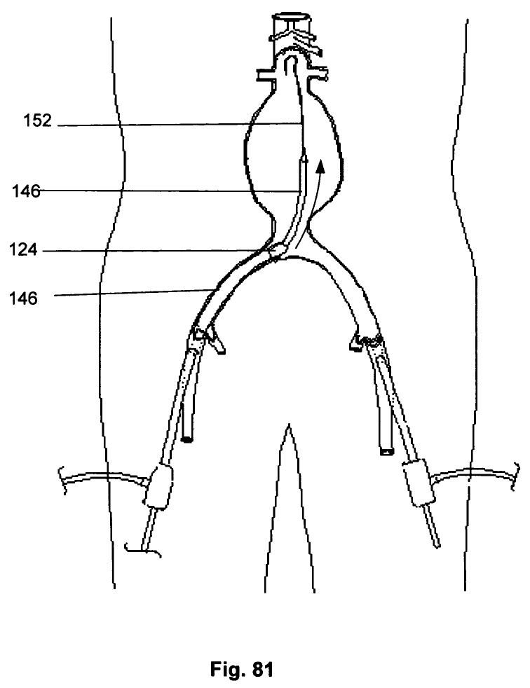

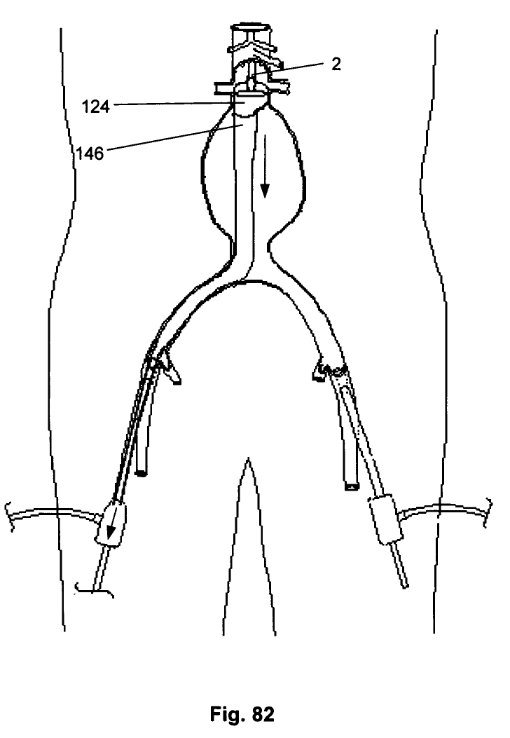

FIG. 80 illustrates a method of deploying the intravascular graft anchoring assembly 2 that can be deployed using the delivery catheter 146 as prepared, for example, as shown in FIGS. 70 or 71. The delivery catheter 146 can be deployed into the first iliac artery 190. The guidewire 152 can be deployed into or toward the neck 166 of the aneurysm 150.

As illustrated in FIG. 81, the intravascular graft anchoring assembly 2 that can be compressed, the delivery catheter 146 and/or the graft 8 can be propelled along the guidewire 152 until the intravascular graft anchoring assembly 2 and the graft 8 are properly positioned, as shown in FIG. 82. FIG. 82 also illustrates that the delivery catheter 146 can begin to be withdrawn, as shown by arrows, leaving the intravascular graft anchoring assembly in the supra-aneurysm and/or suprarenal aorta 188 and exposing the proximal end of the graft body 124.

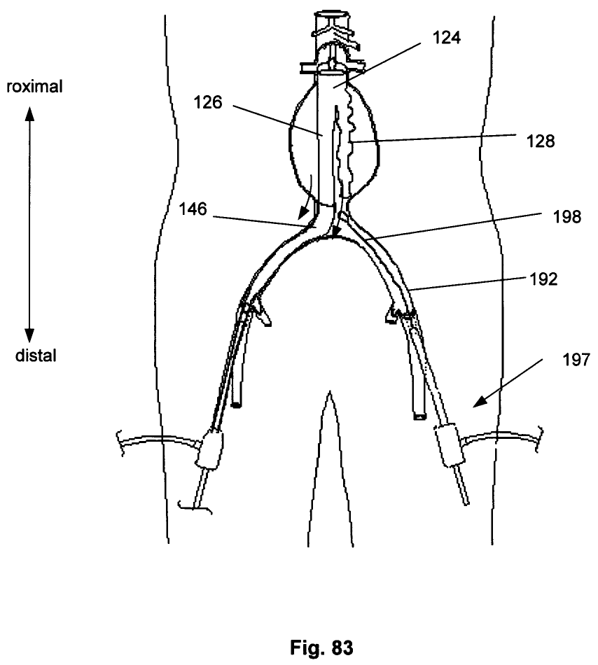

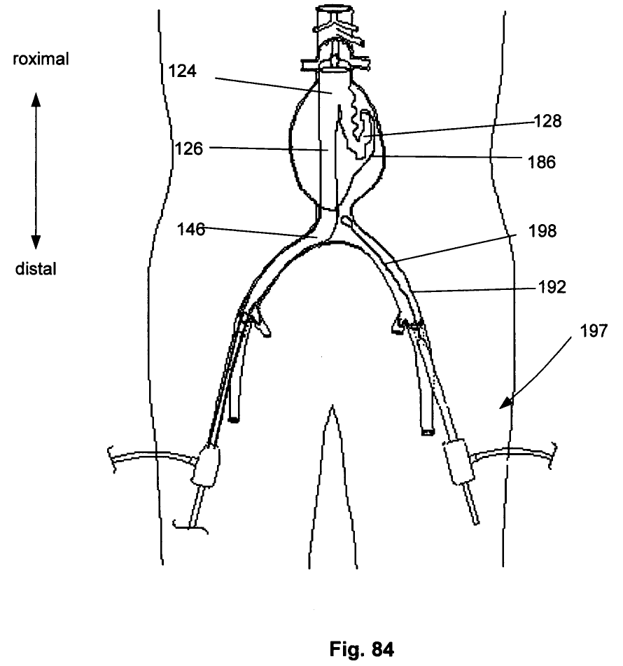

FIG. 83 illustrates a the use of the graft 8 and delivery catheter 146 illustrated in FIG. 71. As the delivery catheter 146 is withdrawn from the aneurysm 150, as shown by arrows, the second graft leg 128 can emerge from the delivery catheter 146 in a potentially corrugated configuration. The open end of the second graft leg 128 can be pointing distally. A snare 198 can be introduced to a location near the open end of the second graft leg. The snare 198 can be introduced from the vascular access device 197 on the second iliac artery 192. The snare 198 can attach to the second graft leg 128 and pull the second graft leg 128 to desired location, for example, as shown in FIG. 79.

FIG. 84 illustrates a the use of the graft 8 and delivery catheter 146 illustrated in FIG. 70. As the delivery catheter 146 is withdrawn from the aneurysm 150, as shown by arrows, the second graft leg 128 can emerge from the delivery catheter 146 in a potentially corrugated configuration. The open end of the second graft leg 128 can be directed proximally or distally. As the delivery catheter 146 is withdrawn from the patient's body, the tether 186 attached to the delivery catheter 146 and the second graft leg 128 can pull the open end of second graft leg 128 to point distally. The snare 198 can be introduced from the vascular access device 197 on the second iliac artery 192. The snare 198 can attach to the second graft leg 128 and/or the tether 186 and pull the second graft leg 128 to desired location, for example, as shown in FIG. 79. The tether 186 can then be detached from the graft 8 and the delivery catheter 146.

It is apparent to one skilled in the art that various changes and modifications can be made to this disclosure, and equivalents employed, without departing from the spirit and scope of the invention. Elements shown with any embodiment are exemplary for the specific embodiment and can be used on other embodiments within this disclosure.

1. A fixation device for implantation in a biological vessel comprising:
a frame comprising a longitudinal axis,
wherein the frame is configured to expand at variable amounts circumferentially with respect to the longitudinal axis.

2. The device of claim 1, wherein the frame comprises a first section and a second section, and wherein the first section remains fixed with respect to the vessel.

3. The device of claim 2, wherein the second section comprises about 180 contiguous degrees of the device.

4. A vascular fixation device comprising:
a first fixation section;
a first arm comprising a first end and a second end, wherein the first end is attached to the first fixation section; and
a second fixation section, wherein the second end of the first arm is attached to the second fixation section.

5. The device of claim 4, further comprising a second arm comprising a first end and a second end, wherein the first end of the second arm is attached to the first fixation section, and wherein the second end of the second arm is a terminus.

6. The device of claim 5, further comprising a third arm extending from the second fixation section.

7. A vascular fixation device comprising:
a first fixation section;
a first arm extending from the first fixation section, wherein the first arm comprises a first end, and wherein the first end of the first arm comprises a terminus; and
a second arm extending from the first fixation section, wherein the second arm comprises a first end, and wherein the first end of the second arm comprises a terminus.

8. The device of claim 7, wherein the first arm extends from the fixation section in a first direction and wherein the second arm extends from the fixation section in a second direction and wherein the first direction is substantially opposite to the second direction.

9. The device of claim 7, further comprising a graft attachment device comprising a first end and a second end, wherein the first end of the graft attachment device is attached to the fixation section.

10. The device of claim 9, wherein the second end of the graft attachment device is attached to a first vascular graft.

11. The device of claim 10, wherein the second end of the graft attachment device is attached to a second vascular graft.

12. The device of claim 9, wherein the first end of the graft attachment device is attached to the fixation section near the vascular wall.

13. The device of claim 9, wherein the graft attachment device is configured to radially expand when the graft attachment device is subject to a force in the direction of the graft.

14. The device of claim 9, wherein the fixation section comprises a tissue anchoring device.

15. The device of claim 14, wherein the tissue anchoring device comprises holes in a surface.

16. The device of claim 14, wherein the tissue anchoring device comprises a spike.

17. The device of claim 14, wherein the tissue anchoring device comprises a tab.

18. The device of claim 17, wherein the tab is directed at least in part into the vascular wall.

19. The device of claim 7, wherein the first arm is longitudinally distanced from the second arm.

20. The device of claim 7, wherein the first arm comprises a first helical section.

21. The device of claim 9, wherein the second arm comprises a second helical section.

22. The device of claim 7, wherein the first arm comprises a first strut, a first member, and a second member, and wherein the first strut comprises a first end and a second end, and wherein the first end of the first strut is attached to the first member and the second end of the first strut is attached to the second member.

23. The device of claim 22, wherein the second arm comprises a second strut, a third member, and a fourth member, and wherein the second strut comprises a first end and a second end, and wherein the first end of the second strut is attached to the third member and the second end of the second strut is attached to the fourth member.

24. The device of claim 7, wherein the first arm rotates less than about 180 degrees around the vascular wall.

25. The device of claim 24, wherein the second arm rotates less than about 180 degrees around the vascular wall.

26. An assembly comprising:
a first device of claim 7,
a device extender comprising a first end and a second end, wherein the first end of the device extender is attached to the first device of claim 7, and
a second device of claim 7, wherein the second end of the device extender is attached to the second device of claim 7.

27. A device for fixing to a vascular wall comprising:
a fixation section;
a first arm extending from a first side of the fixation section;
a second arm extending from a second side of the fixation section; and
a graft attachment device comprising a first end and a second end, wherein the first end of the graft attachment device is attached to the fixation section.

28. The device of claim 27, wherein the first arm extends from the fixation device in a first direction and wherein the second arm extends from the fixation device in a second direction and wherein the first direction is substantially opposite to the second direction.

29. The device of claim 27, wherein the second end of the graft attachment device is attached to a first vascular graft.

30. The device of claim 29, wherein the first vascular graft comprises a bifurcated graft.

31. The device of claim 30, wherein the second end of the graft attachment device is attached to a second vascular graft.

32. The device of claim 27, wherein the first end of the graft attachment device is attached to the fixation section near the vascular wall.

33. The device of claim 27, wherein the graft attachment device is configured to radially expand when the graft attachment device is subject to a force in the direction of the graft.

34. The device of claim 27, wherein the first arm is axially distanced from the second arm.

35. The device of claim 27, wherein the first arm comprises a first helical section.

36. The device of claim 35, wherein the second arm comprises a second helical section.

37. The device of claim 27, wherein the first arm rotates less than about 180 degrees around the vascular wall.

38. The device of claim 37, wherein the second arm rotates less than about 180 degrees around the vascular wall

39. The device of claim 27, wherein the fixation section comprises a tissue anchoring device.

40. The device of claim 39, wherein the tissue anchoring device comprises a surface comprising holes.

41. The device of claim 39, wherein the tissue anchoring device comprises a spike.

42. The device of claim 39, wherein the tissue anchoring device comprises a tab.

43. The device of claim 42, wherein the tab is directed at least in part into the vascular wall.

44. An assembly comprising:
a first device of claim 27,
a device extender comprising a first end and a second end, wherein the first end of the device extender is attached to the first device of claim 27, and
a second device of claim 27, wherein the second end of the device extender is attached to the second device of claim 27.

45. An assembly for fixing to a vascular wall comprising:
an anchor; and
a graft comprising a first end, wherein the graft is attached to the anchor, and
wherein the assembly is configured that when a force is applied pushing the graft away from the anchor then the first end of the graft radially expands.

46. A method of attaching a vascular prosthesis to a vascular wall comprising:
deploying a fixation device in a vessel, wherein the fixation device comprises a fixation section, a first arm extending from the fixation section, and a second arm extending from the fixation section,
attaching a vascular prosthesis to the fixation device.

47. A method of using a vascular prosthesis comprising a first leg, a second leg and a trunk attached to the first leg and the second leg, the method comprising:
deploying the first leg into a first iliac artery,
then extending the trunk across the aneurysm.

48. The method of claim 47, further comprising deploying the second leg into the second iliac artery before extending the trunk.

49. A method of using a vascular prosthesis at a vascular site, the prosthesis comprising a first leg and a second leg, and wherein a bifurcation angle is formed between the first leg and a second leg, the method comprising:
configuring the vascular prosthesis so the bifurcation angle is greater than about 120 degrees,
deploying the vascular prosthesis at the vascular site.

50. The method of claim 49, further comprising causing the bifurcation angle of the vascular prosthesis to decrease, during or after the vascular prosthesis is deployed.
